(19) United States
(12) Patent Application Publication
(10) Pub. No.: US 2010/0041741 A1
(43) Pub. Date: Feb. 18, 2010

(54) SUPPRESSION OF MITOCHONDRIAL OXIDATIVE STRESS

(76) Inventor: John Guy, Gainesville, FL (US)

Correspondence Address: Gregory A. Nelson;Novak Druce & Quigg LLP, 525 Okeechobee Blvd, Suite 1500, West Palm Beach, FL, 33401, US

(21) Appl. No.: 12/446,878

(22) PCT Filed: Oct. 19, 2007
(86) PCT No.: PCT/US07/81922

§ 371 Date: Apr. 23, 2009

Related U.S. Application Data
(60) Provisional application No. 60/862,469, filed on Oct. 23, 2006.

(51) Int. Cl. A61K 31/7088; C12N 15/00; C12N 5/10

(52) U.S. Cl. 514/44 R; 435/320.1; 435/325; 435/366; 435/375

(57) ABSTRACT

Compositions for suppressing and inhibiting mitochondrial oxidative stress. The compositions include a human mitochondrial superoxide dismutase (SOD2) gene. Methods of treating patients suffering from diseases or disorders associated with mitochondrial oxidative stress.

### FIELD OF THE INVENTION

This invention relates to suppression of mitochondrial oxidative stress.

### BACKGROUND

The mitochondrion is a major source of cellular reactive oxygen species (O²⁻), which are formed during electron transport. These reactive oxygen species are capable of preferentially damaging the mitochondrial membranes and proteins, affecting key cell functions, including mitochondrial respiration, which, if altered, leads to increased reactive oxygen species production, mediating lipid peroxidation and DNA damage. Because mitochondrial oxidative phosphorylation (OXPHOS) capacities decline as mitochondrial DNA (mtDNA) damage and mutations accumulate with age, mitochondrial damage and reactive oxygen species generation may act as catalysts for age-related degenerative disease, such as coronary artery disease (CAD). It was hypothesized that free radicals generated within the endothelial and smooth muscle cell environment mediate mitochondrial damage within these cells, establishing a vicious cycle of further reactive oxygen species generation and mitochondrial damage leading to vascular cell dysfunction.

The art is deficient in treating diseases or disorders associated with reactive oxygen intermediates. The present invention fulfills this long-standing need and desire in the art.

### SUMMARY

Compositions for suppressing and inhibiting mitochondrial oxidative stress. The compositions include a human mitochondrial superoxide dismutase (SOD2) gene. Methods of treating patients suffering from diseases or disorders associated with mitochondrial oxidative stress.

In a preferred embodiment an adeno-associated AAV vector comprises a cytomegalovirus enhancer, a promoter, a human mitochondrial superoxide dismutase (SOD2) gene, an internal ribosome entry site (IRES) and a detectable marker gene.

In another preferred embodiment, the cytomegalovirus enhancer, the promoter, the human mitochondrial superoxide dismutase (SOD2) gene, the internal ribosome entry site (IRES) and the detectable marker gene are interposed between inverted terminal repeat sequences. Preferably, the vector is an AAV vector comprising Rep, Cap, inverted terminal repeat (ITR) sequences and the AAV is selected from the group consisting of AAV-1 to AAV-9 serotypes.

In another preferred embodiment, the promoter is a hybrid cytomegalovirus/β-actin promoter.

In another preferred embodiment, a cell comprising an adeno-associated AAV vector comprising: a cytomegalovirus enhancer, a promoter a human mitochondrial superoxide dismutase (SOD2) gene, an internal ribosome entry site (IRES) and a detectable marker gene; wherein the vector is an AAV vector comprising Rep, Cap, inverted terminal repeat (ITR) sequences and the AAV is selected from the group consisting of AAV-1 to AAV-9 serotypes.

In another preferred embodiment, the cytomegalovirus enhancer, the promoter, the human mitochondrial superoxide dismutase (SOD2) gene, the internal ribosome entry site (IRES) and the detectable marker gene are interposed between the inverted terminal repeat sequences.

In another preferred embodiment, the cell is a mammalian cell.

In another preferred embodiment, the cell is isolated from a patient suffering from a disease or disorder associate with abnormal levels of reactive oxygen species comprising: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

In another preferred embodiment, a method of inhibiting mitochondrial oxidative stress in a cell or animal, comprises administering to a cell or animal a nucleic acid comprising an adeno-associated AAV vector comprising: a cytomegalovirus enhancer, a promoter, a human mitochondrial superoxide dismutase (SOD2) gene, an internal ribosome entry site (IRES) and a detectable marker gene; and; expressing the human mitochondrial superoxide dismutase (SOD2) gene in the cell or animal; and, inhibiting mitochondrial oxidative stress in a cell or animal.

In another preferred embodiment, the nucleic acid is administered in an amount sufficient to inhibit mitochondrial oxidative stress in a cell or animal between about 50% up to 100% as compared to an abnormal cell; and, normalizing reactive oxygen species in the abnormal cell to normal cell levels.

In another preferred embodiment, an abnormal cell comprises a cell isolated from a patient that is suffering from a disease or condition caused by reactive oxygen species comprising inflammation, shock, cancer and ischemia/reperfusion injury.

In another preferred embodiment, the abnormal cell is isolated from a patient suffering from a disease or disorder associate with abnormal levels of reactive oxygen species comprising: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

In another preferred embodiment, a method of treating a patient suffering from a disease or disorder associated with enhanced mitochondrial oxidative stress comprises administering to a patient a nucleic acid comprising an adeno-associated AAV vector comprising: a cytomegalovirus enhancer, a promoter, a human mitochondrial superoxide dismutase (SOD2) gene, an internal ribosome entry site (IRES) and a detectable marker gene; and; expressing the a human mitochondrial superoxide dismutase (SOD2) gene in a cell or patient; and, treating the patient suffering from a disease or disorder associated with enhanced mitochondrial oxidative stress.

In another preferred embodiment, the nucleic acid is administered in an amount sufficient to inhibit mitochondrial oxidative stress in the patient between about 50% up to 100% as compared to an abnormal cell; and, normalizing reactive oxygen species in the abnormal cell to normal cell levels.

In another preferred embodiment, the disease or disorder associate with abnormal levels of reactive oxygen species comprises: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

In another preferred embodiment, a method of introducing nucleic acid molecules into mitochondria (intra-mitochondrially) comprises administering to a cell or patient a composition comprising a vector encoding a cytomegalovirus enhancer, a promoter, a human mitochondrial superoxide dismutase (SOD2) gene, an internal ribosome entry site (IRES) and a detectable marker gene; introducing nucleic acid molecules into mitochondria.

In another preferred embodiment, the cytomegalovirus enhancer, the promoter, the human mitochondrial superoxide dismutase (SOD2) gene, the internal ribosome entry site (IRES) and the detectable marker gene are interposed between inverted terminal repeat sequences.

In another preferred embodiment, the vector is an AAV vector comprising Rep, Cap, inverted terminal repeat (ITR) sequences and the AAV is selected from the group consisting of AAV-1 to AAV-9 serotypes.

In another preferred embodiment, an Adenovirus Associated Virion comprises a cytomegalovirus enhancer, a promoter, a human mitochondrial superoxide dismutase (SOD2) gene, an internal ribosome entry site (IRES) and a detectable marker gene.

In another preferred embodiment, the cytomegalovirus enhancer, the promoter, the human mitochondrial superoxide dismutase (SOD2) gene, the internal ribosome entry site (IRES) and the detectable marker gene are interposed between inverted terminal repeat sequences.

Preferably, the vector is an AAV vector comprising Rep, Cap, inverted terminal repeat (ITR) sequences and the AAV is selected from the group consisting of AAV-1 to AAV-9 serotypes and the promoter is a hybrid cytomegalovirus/β-actin promoter.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A-1L show a series of scans of photographs of fluorescence microscopy of ROS. Superoxide anion was undetectable in a normal optic nerve cross section (FIG. 1A) that showed labeling of mitochondria with green labeling (FIGS. 1B, 1C). Three days after sensitization for EAE, red labeling with dihydroethidium revealed superoxide anion in the optic nerve (FIG. 1D). ROS activation was associated with diminished green labeling of mitochondria (FIG. 1E) relative to the normal optic nerve (FIG. 1B). Some colocalization is shown in the merged panel (FIG. 1F). SOD2 gene inoculation attenuated superoxide in the 3-day EAE optic nerve (FIG. 1G) relative to untreated EAE (FIG. 1D) with diminished mitochondrial labeling (FIG. 1H), particularly at foci at which superoxide anion was highly expressed (FIG. 1I). In a longitudinal section of the 6-day EAE optic nerve, red labeling of mitochondria (FIG. 1J) was associated with hydrogen peroxide detected by green labeling with DCFDA (FIG. 1K), often colocalizing with red labeling except at several perivascular foci (arrows) where hydrogen peroxide was highly expressed (FIG. 1L).

FIGS. 2A-2D are scans of photographs showing inflammation. Toluidine blue staining (FIG. 2A) and immunofluorescence labeling with an anti-macrophage antibody (FIG. 2B) show no inflammatory cells in the 3-day EAE optic nerve. In the 30-day EAE optic nerve, inflammatory cells (FIG. 2C, arrows) were identified as macrophages (FIG. 2D).

FIGS. 3A-3F are scans of photographs showing apoptosis. TUNEL-positive cells were seen in the ganglion cell layer of the retina in 3-day EAE animals (FIG. 3A) but not in controls inoculated with the adjuvant (FIG. 3B). In addition, the 3-day EAE optic nerve revealed TUNEL-positive cells (FIG. 3C, arrows). Optic nerves of animals inoculated with the adjuvant were TUNEL negative (FIG. 3D). Colocalization with an anti-oligodendrocyte antibody identified TUNEL-positive cells (arrows) as oligodendrocytes (FIG. 3E). Oligodendrocytes (arrows) in adjuvant-inoculated nerves were TUNEL negative (FIG. 3F).

FIGS. 4A-4U show the modulation of antioxidant genes in acute optic neuritis. Relative to the normal optic nerve head (FIG. 4A, arrows), filling of the optic cup (arrows) and displacement of the peripapillary retina are seen in acute EAE (FIG. 4B, arrows). A ribozyme suppressing SOD2 expression markedly increases optic nerve head swelling in EAE-sensitized mice (FIG. 4C, arrows), whereas SOD2 overexpression suppresses it (FIG. 4D, arrows). Retinas of a normal animal (FIG. 4E) and a 1-month-old EAE animal (FIG. 4F) contrast with the severe loss of the RGC layer in a 1-month EAE animal inoculated with the SOD2 ribozyme (FIG. 4G) and with SOD2 treatment (FIG. 4H). Relative to the normal unmyelinated optic nerve head (FIG. 4I), transmission electron microscopy reveals hydropic degeneration of axons and mitochondria (arrow) in a 1-month EAE animal (FIG. 4J) that is exacerbated by suppressed SOD2 expression with the ribozyme (FIG. 4K, asterisks) and is ameliorated by SOD2 overexpression (FIG. 4L). Compared with the normal retrobulbar optic nerve in which myelinated axons (a) are evident (FIG. 4M), inflammatory cells (IC) and demyelinated axons (a) are seen in a 1-month EAE animal (FIG. 4N). The ribozyme against SOD2 increased axonal loss and myelin loss (FIG. 4O), whereas SOD2 gene transfer ameliorated them (FIG. 4P). Compared with axons and mitochondria of the normal nerve (FIG. 4Q), electron-dense cerium perhydroxide reaction product (arrows) is evident within mitochondria, some swollen with dissolution of cristae, in the optic nerve of a 1-month EAE animal (FIG. 4R). The ribozyme against SOD2 increased hydropic degeneration of mitochondria (arrows) in EAE-sensitized animals even in myelinated axons (FIG. 4S), whereas SOD2 suppressed mitochondrial (arrows) and axonal injury in acute optic neuritis (FIG. 4T). (FIG. 4U) Histogram of myelin fiber area in the normal optic nerve, uninoculated EAE, RzSOD2 treatment in EAE, RzSOD2 treatment in normal unsensitized mice, and SOD2-inoculated eyes compared with treatment with AAV-GFP. It shows the anti-SOD2 ribozyme exacerbated acute optic neuritis, decreasing myelin fiber area by 23% compared with treatment with AAV-GFP in mice sensitized for EAE (P<0.01). Myelin fiber area in mice sensitized for EAE and injected with the anti-SOD2 ribozyme was reduced 41% compared with mice injected with RzSOD2 but not sensitized for EAE (P<0.005). Treatment with AAV-SOD2 was beneficial, with a 46% protective effect on myelin fiber preservation relative to the contralateral eyes treated with AAV-GFP(P<0.01). INL, inner nuclear layer.

FIGS. 5A-5L show SOD2 expression and MRI. (FIG. 5A) Northern blot analysis showing that human SOD2 expression was absent in the mouse cell line infected with AAV-GFP but that human SOD2 mRNA was transcribed after inoculation with AAV-SOD2. GAPDH expression was comparable in both infected cell lines. Western blot showing that relative to the endogenous levels of MnSOD treated with AAV-GFP, MnSOD expression was substantially increased after SOD2 treatment in cultured RGC-5 cells (FIG. 5B) and in the murine optic nerve (FIG. 5C). β-actin expression is shown for protein loading. (FIG. 5D) Compared with treatment with AAV-GFP, MnSOD activity increased almost threefold after SOD2 gene inoculation in transfected RGC-5 cells. (FIG. 5E) Compared with endogenous levels of MnSOD (arrows) in AAV-GFP-inoculated eyes, AAV-SOD2 substantially increased mitochondrial MnSOD immunogold (arrows) in the mouse optic nerve (FIG. 5F). Two weeks after EAE sensitization, MRI showed slightly less swelling of the right optic nerve with SOD2 treatment compared with the AAV-GFP-inoculated left eye (FIG. 5G). One year after sensitization for EAE, optic nerve atrophy seen on the left was suppressed by SOD2 gene inoculation on the right axial MRI (FIG. 5H) and coronal MRI (FIG. 5I). The excised AAV-GFP-inoculated left optic nerves were atrophic compared with right nerves protected by SOD2 3 months (FIG. 5J) and 1 year (FIG. 5K) after EAE sensitization. The classic myelitis of EAE was also evident at 3 months (FIG. 5L). L, left; R, right.

FIGS. 6A-6P shows micrographs of chronic EAE. (FIG. 6A) Excavation of the optic nerve head (arrows) and atrophy of the AAV-GFP-inoculated retrobulbar nerve were seen 3 months after sensitization for EAE. (FIG. 6B) Protection with SOD2 ameliorated cupping of the optic nerve head and atrophy of the retrobulbar nerve. (FIG. 6C) In eyes control injected with AAV-GFP, 1 year after sensitization for EAE, excavation of the optic nerve head was advanced (arrows), extending to the lamina sclerales, and the retrobulbar nerve was markedly atrophic (double arrows). (FIG. 6D) Protection with SOD2 ameliorated cupping of the optic nerve head and atrophy of the retrobulbar nerve even at 1 year. Demyelinated plaques seen at 3 months in control-inoculated nerves (FIG. 6E) were suppressed with SOD2 (FIG. 6F). Cystic spaces (arrows) in the optic nerve seen 1 year after EAE sensitization in control-inoculated nerves (FIG. 6G) were ameliorated with SOD2 treatment (FIG. 6H). RGC loss predominated the chronic stages of EAE (FIG. 6I), contrasting with the preservation of RGCs by SOD2 at 1 year after sensitization for EAE (FIG. 6J). Transmission electron microscopy shows cystic spaces in the nerve fiber layer (NFL) left by degenerating RGCs and an apoptotic cell (FIG. 6K, arrow). SOD2 treatment preserved the NFL and RGCs (FIG. 6L). Axons with swollen mitochondria (arrow) in control (AAV-GFP)-inoculated nerves (FIG. 6M) were ameliorated with SOD2 treatment showing normal axonal mitochondria (arrow), 3 months after sensitization for EAE (FIG. 6N). Degenerating axons, some with aggregation of mitochondria (asterisk), hydropic degeneration, and loss of cristae evidenced ongoing neurodegeneration 1 year after sensitization for EAE (FIG. 6O). These findings were suppressed by SOD2 1 year after sensitization for EAE (FIG. 6P).

FIGS. 7A-7C are histograms showing MRI volume, myelin fiber area, and RGCs. (FIG. 7A) Histogram of MRI optic nerve volume measurements of the right nerve (OD) relative to the left nerve (OS) revealed no differences in EAE animals that received no ocular injections. However, optic nerve swelling characteristic of acute EAE was suppressed by ocular gene injection of SOD2 at 2 weeks (P<0.05), but not at 4 weeks (P>0.05), after sensitization for EAE. Later, optic nerve degeneration was suppressed by SOD2 at 3 months (P<0.02), 4 months (P<0.05), 7 months (P<0.02), and 12 months (P<0.002) after sensitization for EAE. (FIG. 7B) Quantification of myelin fiber area shows the protective effect of SOD2 relative to control gene inoculation with AAV-GFP(P<0.001), the uninoculated EAE nerve (P<0.005), and the normal optic nerve (P<0.05). Myelin fiber loss in EAE relative to normal is also shown (P<0.05). (FIG. 7C) Enumeration of RGCs shows the neuroprotective effect of SOD2 relative to control treatment with AAV-GFP(P<0.005) and uninoculated EAE (P<0.0001). No significant differences were detected between SOD2-treated and normal optic nerve (P>0.05). A one-third loss of RGCs was seen in uninoculated EAE relative to the normal optic nerve (P<0.0005). Treatment with AAV-GFP had a mild protective effect compared with EAE eyes that received no ocular injection (P<0.05).

FIGS. 8A-8I show GFP expression in EAE 1 month after AAV-GFP inoculation. (FIG. 8A) Ganglion cells of the retina expressed GFP. The same retinal section is shown by phase-contrast (FIG. 8B) and merged (FIG. 8C) images. Punctate GFP expression was seen in the optic nerve (FIG. 8D). Inflammatory cells labeled red by the anti-macrophage antibody were also seen in the optic nerve (FIG. 8E). Cells labeled by this antibody did not colocalize with GFP in the merged image (FIG. 8F). GFP labeling in the nerve (FIG. 8G) and oligodendrocytes labeled by the anti-oligodendrocyte antibody (FIG. 8H) did not colocalize in the merged panel (FIG. 8I).

FIGS. 9A-9B are schematic illustrations showing the control adeno-associated viral (AAV) vector plasmid (pTR-UF12) (FIG. 9A) and the AAV containing the superoxide dismutase gene (SOD2) (FIG. 9B). Immunoblots of mitochondrial SOD (FIG. 9C) show that, relative to uninfected Leber hereditary optic neuropathy cells (lane 1) or controls infected with AAV-green fluorescent protein (GFP) (lane 2), manganese SOD (MnSOD) (24 kDa) is increased in cybrid cell cultures infected with AAV-SOD2 (lane 3). Expression of β-actin (42 kDa) is relatively comparable in each of the 3 lanes. CBA indicates chicken β-actin; CMV, cytomegalovirus enhancer; IRES, internal ribosomal entry site; and iTR, inverted terminal repeat.

FIGS. 10A-10D are micrographs showing decreased superoxide-induced dihydroethidium (DHE) fluorescence with adeno-associated viral vector containing the superoxide dismutase gene (AAV-SOD2) (FIG. 10A) relative to AAV-green fluorescent protein (GFP) infection (FIG. 10B), after 1 day in the galactose medium. After 2 days in galactose medium, decreased DHE fluorescence is also evident with AAV-SOD2 infection (FIG. 10C) relative to AAV infection (FIG. 10D) (original magnification ×100). The histogram (FIG. 10E) shows that the mean±SD intensity of superoxide-induced DHE fluorescence is diminished with AAV-SOD2 infection relative to infection with AAV-GFP.

FIGS. 11A-11D are micrographs of TUNEL (terminal deoxynucleotidyl transferase-mediated biotin-deoxyuridine triphosphate nick-end labeling) fluorescence show decreased TUNEL-positive cells with adeno-associated viral vector containing the superoxide dismutase gene (AAV-SOD2) (FIG. 11A) relative to AAV-green fluorescent protein (GFP) infection (FIG. 11B) after 1 day in the galactose medium. After 2 days in galactose medium, a decrease in TUNEL-positive cells is also evident with AAV-SOD2 infection (FIG. 11C) relative to AAV infection (FIG. 11D) (original magnification ×100). The histogram (FIG. 11E) shows that the mean±SD intensity of TUNEL-induced fluorescence is diminished with AAV-SOD2 infection relative to infection with AAV-GFP.

FIGS. 12A-12B are micrographs showing an increase in Leber hereditary optic neuropathy (LHON) cell survival with adeno-associated viral vector containing the superoxide dismutase gene (AAV-SOD2) treatment (FIG. 12A) relative to AAV-green fluorescent protein (GFP) infection (FIG. 12B) after 2 days in galactose medium (original magnification ×100). The histogram (FIG. 12C) shows that the mean±SD LHON cell survival is increased with AAV-SOD2 relative to AAV-GFP infection after 2 and 3 days of growth in the galactose medium (FIG. 12C).

### DETAILED DESCRIPTION

The invention comprises a vector encoding the human mitochondrial superoxide dismutase (SOD2) gene. The expression of the human mitochondrial superoxide dismutase (SOD2) gene in an abnormal cell suppresses mitochondrial oxidative stress. Methods of treating patients comprise expression of the a human mitochondrial superoxide dismutase (SOD2) gene in cells which suppresses mitochondrial oxidative stress.

### Definitions

Prior to setting forth the invention, the following definitions are provided:

As used herein, the singular forms “a”, “an” and “the” include plural referents unless the context clearly dictates otherwise.

As used herein, the term “oxidative stress” refers to pathophysiological effects of reactive oxygen species, such as H₂O₂, superoxide, peroxynitrite, all derivatives of these and other reactive oxygen species, on normal cellular function. The target of oxidative stresses may be proteins, antigens, lipids, RNA, DNA or any other cellular component.

As used herein, the term “antioxidant treatment” refers to any nucleic acid, protein, organic or inorganic substances that interact with reactive oxygen species to nullify their pathophysiological effects. In a preferred embodiment a nucleic acid and products there of are: a human mitochondrial superoxide dismutase (SOD2) gene and an anti-human mitochondrial superoxide dismutase (RzSOD2) ribozyme.

As used herein, the term “MtDNA damage” generally refers to any type of lesion (i.e. base alterations, apurinic sites, strand breaks, adduct formation, etc.) or mtDNA length mutation (deletions, insertions, and duplications) that can potentially be detected either directly by QPCR (by blocking the polymerase, or resulting in a QPCR product of size different than anticipated, i.e. mtDNA length mutations), or in concert with an enzymatic action (i.e. DNA can be treated with FAPY glycosylase before QPCR to detect 8-oxo-G).”

A person having ordinary skill in this art would recognize that measurement of mitochondrial DNA damage is only one potential method to determine oxidative stress. Any “downstream” or resultant effect of mitochondrial DNA damage will reflect the same disease process. For example, measurement of mitochondrial protein production, changes in mitochondrial oxidative phosphorylation or changes in mitochondrial ATP production would accomplish the same goal.

As used herein, a “pharmaceutically acceptable” component is one that is suitable for use with humans and/or animals without undue adverse side effects (such as toxicity, irritation, and allergic response) commensurate with a reasonable benefit/risk ratio.

The term “DNA construct” and “vector” are used herein to mean a purified or isolated polynucleotide that has been artificially designed and which comprises at least two nucleotide sequences that are not found as contiguous nucleotide sequences in their natural environment.

The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides, and may be chemically modified by such means as methylation or the inclusion of protecting groups or cap- or tail structures. Single or double stranded DNA or RNA and linear or circular. Single stranded DNA can be used for expression and circular RNA can also be used for expression.

As used interchangeably herein, the terms “oligo-nucleotides”, “polynucleotides”, and “nucleic acids” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, all as described herein.

The phrase “having a length of N bases” or “having a length of N nucleotides” is used herein to describe lengths along a single nucleotide strand, of a nucleic acid molecule, consisting of N individual nucleotides.

As used herein, the term “bind”, refers to an interaction between the bases of an oligonucleotide which is mediated through base-base hydrogen bonding. One type of binding is “Watson-Crick-type” binding interactions in which adenine-thymine (or adenine-uracil) and guanine-cytosine base-pairs are formed through hydrogen bonding between the bases. An example of this type of binding is the binding traditionally associated with the DNA double helix.

As used herein, the term “oligonucleotide” refers to a polynucleotide formed from naturally occurring bases and pentofuranosyl groups joined by native phosphodiester bonds. This term effectively refers to naturally occurring species or synthetic species formed from naturally occurring subunits or their close homologs. The term “oligonucleotide” may also refer to moieties which function similarly to naturally occurring oligonucleotides but which have non-naturally occurring portions. Thus, oligonucleotides may have altered sugar moieties or intersugar linkages. Exemplary among these are the phosphorothioate and other sulfur-containing species which are known for use in the art. In accordance with some preferred embodiments, at least some of the phosphodiester bonds of the oligonucleotide have been substituted with a structure which functions to enhance the ability of the compositions to penetrate into the region of cells where the RNA or DNA whose activity to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with other structures which are, at once, substantially non-ionic and non-chiral, or with structures which are chiral and enantiomerically specific. Persons of ordinary skill in the art will be able to select other linkages for use in practice of the invention.

Oligonucleotides may also include species which include at least some modified base forms. Thus, purines and pyrimidines other than those normally found in nature may be so employed. Similarly, modifications on the pentofuranosyl portion of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)ₙNH₂ or O(CH₂)ₙCH₃ where n is from 1 to about 10, and other substituents having similar properties.

As used herein, the term “administering a molecule to a cell” (e.g., an expression vector, nucleic acid, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A “vector” (sometimes referred to as gene delivery or gene transfer “vehicle”) refers to a macromolecule or complex of molecules comprising a polynucleotide to be delivered to a host cell, either in vitro or in vivo. The “vector” can be any nucleic acid- and/or viral-based technique used to deliver a desired nucleic acid. The polynucleotide to be delivered may comprise a coding sequence of interest in gene therapy. Vectors include, for example, viral vectors (such as adenoviruses (“Ad”), adeno-associated viruses (AAV), and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

By “nucleic acid” is meant both RNA and DNA including: cDNA, genomic DNA, plasmid DNA or condensed nucleic acid, nucleic acid formulated with cationic lipids, nucleic acid formulated with peptides, cationic polymers, RNA or mRNA. In a preferred embodiment, the nucleic acid administered is a plasmid DNA which constitutes a “vector.” The nucleic acid can be, but is not limited to, a plasmid DNA vector with a eukaryotic promoter which expresses a protein with potential therapeutic action.

As used herein, the term a “plasmid” refers to a construct made up of genetic material (i.e., nucleic acids). It includes genetic elements arranged such that an inserted coding sequence can be transcribed in eukaryotic cells. In this case, a preferred embodiment comprises a mitochondrial targeting sequence, a mitochondrial gene operably linked to a mitochondrial promoter. Also, while the plasmid may include a sequence from a viral nucleic acid, such viral sequence preferably does not cause the incorporation of the plasmid into a viral particle, and the plasmid is therefore a non-viral vector. Preferably, a plasmid is a closed circular DNA molecule. The enhancer/promoter region of an expression plasmid will determine the levels of expression. Most of the gene expression systems designed for high levels of expression contain the intact human cytomegalovirus (CMV) immediate early enhancer/promoter sequence. However, down-regulation of the CMV promoter over time has been reported in tissues. The hypermethylation of the CMV promoter, as observed when incorporated into retroviral vectors, has not been observed for episomal plasmids in vivo. Nevertheless, the CMV promoter silencing could be linked to its sensitivity to reduced levels of the transcription factor NF-κB. The activity of the CMV promoter has also been shown to be attenuated by various cytokines including interferons (α and β), and tumor necrosis factor (TNF-α). In order to prolong expression in vivo and ensure specificity of expression in desired tissues, tissue-specific enhancer/promoters have been incorporated in expression plasmids. The chicken skeletal alpha actin promoter has been shown to provide high levels of expression (equivalent to the ones achieved with a CMV-driven construct) for several weeks in non-avian striated muscles.

Additional genetic sequences in the expression plasmids can be added to influence the stability of the messenger RNA (mRNA) and the efficiency of translation. The 5′ untranslated region (5′ UTR) is known to effect translation and it is located between the cap site and the initiation codon. The 5′ UTR should ideally be relatively short, devoid of strong secondary structure and upstream initiation codons, and should have an initiation codon AUG within an optimal local context. The 5′ UTR can also influence RNA stability, RNA processing and transcription. In order to maximize gene expression by ensuring effective and accurate RNA splicing, one or more introns can be included in the expression plasmids at specific locations. The possibility of inefficient and/or inaccurate splicing can be minimized by using synthetic introns that have idealized splice junction and branch point sequences that match the consensus sequence. Another important sequence within a gene expression system is the 3′ untranslated region (3′ UTR), a sequence in the mRNA that extends from the stop codon to the poly(A) addition site. The 3′ UTR can influence mRNA stability, translation and intracellular localization. The skeletal muscle .alpha.-actin 3′ UTR has been shown to stabilize mRNA in muscle tissues thus leading to higher levels of expression as compared to other 3′ UTR. This 3′ UTR appears to induce a different intracellular compartmentalization of the produced proteins, preventing the effective trafficking of the proteins to the secretory pathway and favoring their perinuclear localization. One of the attractive features of plasmid expression systems is the possibility to express multiple genes from a single construct.

Viral “packaging” as used herein refers to a series of intracellular events that results in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsidation or other coating of the nucleic acid. Thus, when a suitable vector plasmid is introduced into a packaging cell line under appropriate conditions, it can be replicated and assembled into a viral particle. Viral “rep” and “cap” gene products, found in many viral genomes, are gene products encoding replication and encapsidation proteins, respectively. A “replication-defective” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain gene products encoding the missing functions which can be supplied in trans). For example, such packaging gene products can be stably integrated into a replicon of the packaging cell line or they can be introduced by transfection with a “packaging plasmid” or helper virus carrying gene products encoding the missing functions.

A “detectable marker gene” is a gene that allows cells carrying the gene to be specifically detected (e.g., distinguished from cells which do not carry the marker gene). A large variety of such marker gene products are known in the art. Preferred examples thereof include detectable marker gene products which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker gene products have been described, including bifunctional (i.e. positive/negative) markers (see, e.g., WO 92/08796, published May 29, 1992, and WO 94/28143, published Dec. 8, 1994). Such marker gene products can provide an added measure of control that can be advantageous in gene therapy contexts.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented.

As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Treatment of Disease or Disorders Associated with Mitochondrial Oxidative Stress

In a preferred embodiment an adeno-associated AAV vector comprises a cytomegalovirus enhancer, a promoter, a human mitochondrial superoxide dismutase (SOD2) gene, an internal ribosome entry site (IRES) and a detectable marker gene.

In another preferred embodiment, the cytomegalovirus enhancer, the promoter, the human mitochondrial superoxide dismutase (SOD2) gene, the internal ribosome entry site (IRES) and the detectable marker gene are interposed between inverted terminal repeat sequences. Preferably, the vector is an AAV vector comprising Rep, Cap, inverted terminal repeat (ITR) sequences and the AAV is selected from the group consisting of AAV-1 to AAV-9 serotypes.

In another preferred embodiment, the promoter is a hybrid cytomegalovirus/β-actin promoter.

In another preferred embodiment, a cell comprising an adeno-associated AAV vector comprising: a cytomegalovirus enhancer, a promoter a human mitochondrial superoxide dismutase (SOD2) gene, an internal ribosome entry site (IRES) and a detectable marker gene; wherein the vector is an AAV vector comprising Rep, Cap, inverted terminal repeat (ITR) sequences and the AAV is selected from the group consisting of AAV-1 to AAV-9 serotypes.

In another preferred embodiment, the cytomegalovirus enhancer, the promoter, the human mitochondrial superoxide dismutase (SOD2) gene, the internal ribosome entry site (IRES) and the detectable marker gene are interposed between the inverted terminal repeat sequences.

In another preferred embodiment, the cell is a mammalian cell.

In another preferred embodiment, the cell is isolated from a patient suffering from a disease or disorder associate with abnormal levels of reactive oxygen species comprising: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

In another preferred embodiment, a method of inhibiting mitochondrial oxidative stress in a cell or animal, comprises administering to a cell or animal a nucleic acid comprising an adeno-associated AAV vector comprising: a cytomegalovirus enhancer, a promoter, a human mitochondrial superoxide dismutase (SOD2) gene, an internal ribosome entry site (IRES) and a detectable marker gene; and; expressing the human mitochondrial superoxide dismutase (SOD2) gene in the cell or animal; and, inhibiting mitochondrial oxidative stress in a cell or animal.

In another preferred embodiment, the nucleic acid is administered in an amount sufficient to inhibit mitochondrial oxidative stress in a cell or animal between about 50% up to 100% as compared to an abnormal cell; and, normalizing reactive oxygen species in the abnormal cell to normal cell levels.

In another preferred embodiment, an abnormal cell comprises a cell isolated from a patient that is suffering from a disease or condition caused by reactive oxygen species comprising inflammation, shock, cancer and ischemia/reperfusion injury.

In another preferred embodiment, the abnormal cell is isolated from a patient suffering from a disease or disorder associate with abnormal levels of reactive oxygen species comprising: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

In another preferred embodiment, a method of treating a patient suffering from a disease or disorder associated with enhanced mitochondrial oxidative stress comprises administering to a patient a nucleic acid comprising an adeno-associated AAV vector comprising: a cytomegalovirus enhancer, a promoter, a human mitochondrial superoxide dismutase (SOD2) gene, an internal ribosome entry site (IRES) and a detectable marker gene; and; expressing the a human mitochondrial superoxide dismutase (SOD2) gene in a cell or patient; and, treating the patient suffering from a disease or disorder associated with enhanced mitochondrial oxidative stress.

In another preferred embodiment, the nucleic acid is administered in an amount sufficient to inhibit mitochondrial oxidative stress in the patient between about 50% up to 100% as compared to an abnormal cell; and, normalizing reactive oxygen species in the abnormal cell to normal cell levels.

In another preferred embodiment, the disease or disorder associate with abnormal levels of reactive oxygen species comprises: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

In another preferred embodiment, a method of introducing nucleic acid molecules into mitochondria (intra-mitochondrially) comprises administering to a cell or patient a composition comprising a vector encoding a cytomegalovirus enhancer, a promoter, a human mitochondrial superoxide dismutase (SOD2) gene, an internal ribosome entry site (IRES) and a detectable marker gene; introducing nucleic acid molecules into mitochondria.

In another preferred embodiment, the cytomegalovirus enhancer, the promoter, the human mitochondrial superoxide dismutase (SOD2) gene, the internal ribosome entry site (IRES) and the detectable marker gene are interposed between inverted terminal repeat sequences.

In another preferred embodiment, the vector is an AAV vector comprising Rep, Cap, inverted terminal repeat (ITR) sequences and the AAV is selected from the group consisting of AAV-1 to AAV-9 serotypes.

In another preferred embodiment, an Adenovirus Associated Virion comprises a cytomegalovirus enhancer, a promoter, a human mitochondrial superoxide dismutase (SOD2) gene, an internal ribosome entry site (IRES) and a detectable marker gene.

In another preferred embodiment, the cytomegalovirus enhancer, the promoter, the human mitochondrial superoxide dismutase (SOD2) gene, the internal ribosome entry site (IRES) and the detectable marker gene are interposed between inverted terminal repeat sequences.

Preferably, the vector is an AAV vector comprising Rep, Cap, inverted terminal repeat (ITR) sequences and the AAV is selected from the group consisting of AAV-1 to AAV-9 serotypes and the promoter is a hybrid cytomegalovirus/β-actin promoter.

The present invention is intended to be used in the medical field to treat, prevent, or alleviate the symptoms associated with a ROS, associated disease or disorder or reduce the expression of such disease or disorder. Such a disease or disorder refers to a condition of an individual that results at least in part from the production of or exposure to free radicals, particularly oxyradicals, and other “ROS” in vivo. Even though there is only a few if any pathological conditions that are monofactorial, there is an increasing body of literature and knowledge related to the involvement of ROS in disease etiology. For these reasons, the term “ROS associated disease” encompasses pathological states that are recognized in the art as being conditions wherein damage from ROS is believed to contribute to the pathology of the disease state, or wherein administration of a free radical inhibitor (e.g., desferrioxamine), scavenger (e.g., tocopherol, glutathione), or catalyst (e.g., SOD. catalase) is shown to produce a detectable benefit by decreasing symptoms. increasing survival, or providing other detectable clinical benefits in treating or preventing the pathological state. For example but not limiting, the disease states discussed herein are considered ROS-associated diseases (e.g., ischemic reperfusion injury, inflammatory diseases, systemic lupus erythematosis, myocardial infarction, stroke, traumatic hemorrhage, spinal cord trauma, Crohn's disease, autoimmune diseases (e.g., rheumatoid arthritis, diabetes), cataract formation, uveitis, emphysema, gastric ulcers, oxygen toxicity, neoplasia, undesired cell apoptosis, radiation sickness. Further, many inflammatory diseases or disorders will benefit of the present invention, since it is known that ROS intervene in the process of inflammation. For example, the “oxidative burst” of activated neutrophils produces abundant superoxide radical, which is believed to be an essential factor in producing the cytotoxic effect of activated neutrophils. Further, since neutrophils are involved in the early mortality of any grafted or transplanted tissue or cell, an antioxidant would increase the early survival of transplanted or grafted cells, which is critical for the success of transplantation.

ROS can initiate a wide range of toxic oxidative reactions. These include initiation of lipid peroxidation, direct inhibition of mitochondrial respiratory chain enzymes, inactivation of glyceraldehyde-3-phosphate dehydrogenase, inhibition of membrane sodium/potassium ATPase activity, inactivation of membrane sodium channels, and other oxidative modifications of proteins.

ROS (e.g., superoxide, peroxynitrite, hydroxyl radical, and hydrogen peroxide) are all potential reactants capable of initiating DNA single-strand breakage, with subsequent activation of the nuclear enzyme poly(ADP-ribose) synthetase, leading to eventual severe energy depletion of the cells and necrotic-type cell death. Antioxidant treatment inhibits the activation of poly(ADP-ribose) synthetase and prevents the organ injury associated with shock, inflammation, and ischemia/reperfusion.

Leber Hereditary Optic Neuropathy (LHON) was the first disease for which a mtDNA point mutation was identified. LHON usually presents as a bilateral loss of central vision that typically progresses over weeks without pain, until bilateral scotomas remain. The mean age of onset is in the mid-20's, although the range is extremely broad. Initially, the optic disc may be swollen and the peripapillary retinal nerve fiber layer edematous, then the optic disc atrophies. A common feature during the acute phase of LHON is peripapillary microangiopathy, which was first described by Leber in 1871. Histopathology of end stage nerves shows degeneration and secondary demyelination that likely limits spontaneous recovery of vision in 90% of patients with the G11778A point mutation. The pattern visual evoked potential (VEP) is affected in the early stages of LHON and becomes extinguished at the atrophic stage, indicating the loss of function of retinal ganglion cells. Nevertheless, electroretinograms (ERG) remain normal, suggesting the maintenance of photoreceptor cells, bipolar cells and the retinal pigment epithelium. Though LHON is typically monosymptomatic and does not limit life-span, in early onset cases (2-4 years), other organ systems are involved, and are characterized by muscle weakness, general dystonic rigidity, impaired speech and intelligence and short stature.

Most LHON cases are associated with mutations in one of three mitochondrial genes for subunits of NADH ubiquinone oxidoreductase which is complex I of the mitochondrial respiratory chain. This enzyme contains 7 subunits encoded by mtDNA that are intimately associated with the inner mitochondrial membrane and 35 subunits that are encoded by nuclear DNA and imported into the organelle. The connection between LHON and mtDNA was firmly established in 1988, when Wallace and colleagues reported a homoplasmic nucleotide transition from guanosine to adenosine at position 11778, which results in an arginine to histidine substitution in ND4, a subunit of complex I. Since then, several other mutations in genes for NADH dehydrogenase, cytochrome b, cytochrome oxidase or ATP synthase subunits have been identified that also cause familial LHON. Approximately 50% of LHON patients have the G11778A mutation, 20% have the G3460A mutation, which affects the ND1 gene, and 10% have T14484C in the ND6 gene. These three mutations are considered the primary causes of LHON, and each presents a significant risk of blindness. Nevertheless, LHON shows incomplete penetrance and only about 50% of males and 10% of females in LHON families lose vision. In a minority of cases, lack of penetrance can be attributed to heteroplasmy: loss of vision is rare unless more than 70% of the mtDNA population carries the mutation. Heteroplasmy cannot explain the gender bias. Therefore modifier genes leading to physiological or behavioral differences have been offered as possible explanations for the gender bias.

In a preferred embodiment, a composition comprises an adeno-associated AAV vector comprising: a cytomegalovirus enhancer, a promoter, a human mitochondrial superoxide dismutase (SOD2) gene, an internal ribosome entry site (IRES) and a detectable marker gene.

In another preferred embodiment, the nucleic acid comprises one or more genes encoding SOD2 and/or AAV-RzSOD2.

In another preferred embodiment, the adeno-associated AAV vector comprises Rep, Cap, inverted terminal repeat (ITR) sequences and the AAV is selected from the group consisting of AAV-1 to AAV-9 serotypes.

In another preferred embodiment, the promoter is a hybrid cytomegalovirus/β-actin promoter.

In another preferred embodiment, an adeno-associated AAV vector comprises a cytomegalovirus enhancer, a promoter an anti-human mitochondrial superoxide dismutase (RzSOD2) ribozyme, an internal ribosome entry site (IRES) and a detectable marker gene.

In another preferred embodiment, the AAV is selected from the group consisting of AAV-1 to AAV-9 serotypes. The AAV genome can comprise Rep and Cap genes from other AAV serotypes and/or the AAV can be pseudotyped.

The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions which carry the encapsidation functions: the left-hand part of the genome, which contains the rep gene involved in viral replication and expression of the viral genes; and the right-hand part of the genome, which contains the cap gene encoding the capsid proteins of the virus.

The use of vectors derived from the AAVs for transferring genes in vitro and in vivo has been described (see WO 91/18088; WO 93/09239; U.S. Pat. No. 4,797,368, U.S. Pat. No. 5,139,941, EP 488 528). These publications describe various AAV-derived constructs in which the rep and/or cap genes are deleted and replaced by a gene of interest, and the use of these constructs for transferring the said gene of interest in vitro (into cultured cells) or in vivo, (directly into an organism). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques. The invention also relates, therefore, to an AAV-derived recombinant virus whose genome encompasses a sequence encoding a nucleic acid encoding a mitochondrial gene and a mitochondrial targeting sequence, flanked by the AAV ITRs. The invention also relates to a plasmid encompassing a sequence encoding a nucleic acid encoding a desired gene flanked by two ITRs from an AAV. Such a plasmid can be used as it is for transferring the nucleic acid sequence, with the plasmid, where appropriate, being incorporated into a liposomal vector (pseudo-virus).

### Other Vectors

In other preferred embodiments, vectors delivering gene payloads for the treatment of mitochondrial disorders comprise viral and non-viral vectors are used to transduce the mitochondria.

Retrovirus vectors: In another preferred embodiment the mitochondrial genes can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; EP 453242, EP178220; Bernstein et al. Genet. Eng. 7 (1985) 235; McCormick, BioTechnology 3 (1985) 689; International Patent Publication No. WO 95/07358, published Mar. 16, 1995, by Webster, K. A., Kubasiak, L. A., Prentice, H. and Bishopric, N. H.: Stable germline transmission of a hypoxia-activated molecular gene switch. From the double helix to molecular medicine, (ed. W. J. Whelan et al.), Oxford University Press, (2003); and Kuo et al., 1993, Blood 82:845. The retroviruses are integrating viruses which infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are disclosed in WO95/02697.

In general, in order to construct recombinant retroviruses containing a nucleic acid sequence, a plasmid is constructed which contains the LTRs, the encapsidation sequence and the coding sequence. This construct is used to transfect a packaging cell line, which cell line is able to supply in trans the retroviral functions which are deficient in the plasmid. In general, the packaging cell lines are thus able to express the gag, pol and env genes. Such packaging cell lines have been described in the prior art, in particular the cell line PA317 (U.S. Pat. No. 4,861,719); the PsiCRIP cell line (WO90/02806) and the GP⁺envAm-12 cell line (WO89/07150). In addition, the recombinant retroviral vectors can contain modifications within the LTRs for suppressing transcriptional activity as well as extensive encapsidation sequences which may include a part of the gag gene (Bender et al., J. Virol. 61 (1987) 1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are prepared to destroy the viral packaging signal, but retain the structural genes required to package the co-introduced virus engineered to contain the heterologous gene and the packaging signals. Thus, the viral particles that are produced are not capable of producing additional virus. Targeted gene delivery is described in International Patent Publication WO 95/28494, published October 1995.

Lentiviral Vectors: lentiviruses include members of the bovine lentivirus group, equine lentivirus group, feline lentivirus group, ovinecaprine lentivirus group and primate lentivirus group. The development of lentiviral vectors for gene therapy has been reviewed in Klimatcheva et al., 1999, Frontiers in Bioscience 4: 481-496. The design and use of lentiviral vectors suitable for gene therapy is described, for example, in U.S. Pat. No. 6,207,455, issued Mar. 27, 2001, and U.S. Pat. No. 6,165,782, issued Dec. 26, 2000. Examples of lentiviruses include, but are not limited to, HIV-1, HIV-2, HIV-1/HIV-2 pseudotype, HIV-1/SIV, FIV, caprine arthritis encephalitis virus (CAEV), equine infectious anemia virus and bovine immunodeficiency virus. HIV-1 is preferred.

Autonomous parvoviruses are small DNA viruses that replicate autonomously in rapidly dividing cells. The genomes of autonomous parvoviruses do not integrate, at least not at a detectable level. Autonomous parvovirus genomes are single-stranded DNA molecules about 5 kilobases (kb) in size. The genomes are organized such that the NS gene encoding the nonstructural polypeptides NS1 and NS2 is located on the left side of the genome and the VP gene encoding the structural polypeptides required for capsid formation are on the right side of the genome. Expression of the nonstructural polypeptides is controlled by a transcription control sequence called P4 in most parvoviruses, which is located at about map unit position 4 of the genome (assuming the entire genome is 100 map units and numbering is from left to right). Expression of the structural polypeptides is controlled by a transcription control sequence called P38, P39 or P40 in most parvoviruses, which is located at about map unit position 38 to about 40, depending on the autonomous parvovirus. NS1 serves as a trans-activator of the latter transcription control sequence. NS1 is also essential for virus replication and appears to be the primary mediator of parvovirus cytotoxicity, particularly against tumor cells. Autonomous parvovirus genomes also have inverted repeat sequences (i.e., palindromes) at each end which contain essential signals for replication and encapsidation of the virus. There have been several studies on the mechanistics of autonomous parvovirus replication, gene expression, encapsidation, and cytotoxicity. See, for example, Sinkovics, pp. 1281-1290, 1989, Anticancer Res., Vol 9.

Suitable autonomous parvovirus nucleic acid sequences include, but are not limited to, LuIII parvovirus (LuIII), minute virus of mice (MVM; e.g., MVMi and MVMP), hamster parvovirus (e.g., H1), feline panleukopenia virus, canine parvovirus, porcine parvovirus, latent rat virus, mink enteritis virus, human parvovirus (e.g., B19), bovine parvovirus, and Aleutian mink disease parvovirus nucleic acid sequences. LuIII parvovirus is a parvovirus of unknown origin that was isolated as a contaminant of a substrain of human permanent cell line Lu106. The LuIII parvovirus exhibits high infectivity.

Non-viral Vectors: alternatively, the vector can be introduced in vivo as nucleic acid free of transfecting excipients, or with transfection facilitating agents, e.g., lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker [Feigner, et. al., Proc. Natl. Acad. Sci. U.S.A. 84:7413-7417 (1987); see Mackey, et al., Proc. Natl. Acad. Sci. U.S.A. 85:8027-8031 (1988); Ulmer et al., Science 259:1745-1748 (1993)]. The use of cationic lipids may promote encapsulation of negatively charged nucleic acids, and also promote fusion with negatively charged cell membranes [Feigner and Ringold, Science 337:387-388 (1989)]. Particularly useful lipid compounds and compositions for transfer of nucleic acids are described in International Patent Publications WO95/18863 and WO96/17823, and in U.S. Pat. No. 5,459,127. Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

Naked DNA vectors can be introduced into the desired host cells by methods known in the art, e.g., transfection, electroporation, microinjection, transduction, cell fusion, DEAE dextran, calcium phosphate precipitation, use of a gene gun, or use of a DNA vector transporter [see, e.g., Wu et al., J. Biol. Chem. 267:963-967 (1992); Wu and Wu, J. Biol. Chem. 263:14621-14624 (1988); Williams et al., Proc. Natl. Acad. Sci. USA 88:2726-2730 (1991)]. Receptor-mediated DNA delivery approaches can also be used [Curiel et al., Hum. Gene Ther. 3:147-154 (1992); Wu and Wu, J. Biol. Chem. 262:4429-4432 (1987)]. Methods for formulating and administering naked DNA to mammalian muscle tissue are disclosed in U.S. Pat. Nos. 5,580,859 and 5,589,466, the contents of which are incorporated herein by reference.

### Mitochondrial Targeting

Mitochondria contain the molecular machinery for the conversion of energy from the breakdown of glucose into adenosine triphosphate (ATP). The energy stored in the high energy phosphate bonds of ATP is then available to power cellular functions. Mitochondria are mostly protein, but some lipid, DNA and RNA are present. These generally spherical organelles have an outer membrane surrounding an inner membrane that folds (cristae) into a scaffolding for oxidative phosphorylation and electron transport enzymes. Most mitochondria have flat shelf-like cristae, but those in steroid secreting cells may have tubular cristae. The mitochondrial matrix contains the enzymes of the citric acid cycle, fatty acid oxidation and mitochondrial nucleic acids.

Mitochondrial DNA is double stranded and circular. Mitochondrial RNA comes in the three standard varieties; ribosomal, messenger and transfer, but each is specific to the mitochondria. Some protein synthesis occurs in the mitochondria on mitochondrial ribosomes that are different than cytoplasmic ribosomes. Other mitochondrial proteins are made on cytoplasmic ribosomes with a signal peptide that directs them to the mitochondria. The metabolic activity of the cell is related to the number of cristae and the number of mitochondria within a cell. Cells with high metabolic activity, such as heart muscle, have many well developed mitochondria. New mitochondria are formed from preexisting mitochondria when they grow and divide. The inner membranes of mitochondria contain a family of proteins of related sequence and structure that transport various metabolites across the membrane. Their amino acid sequences have a tripartite structure, made up of three related sequences about 100 amino acids in length. The repeats of one carrier are related to those present in the others and several characteristic sequence features are conserved throughout the family.

Targeting of specific polynucleotides to organelles can be accomplished by modifying the disclosed compositions to express specific organelle targeting signals. These sequences target specific organelles, but in some embodiments the interaction of the targeting signal with the organelle does not occur through a traditional receptor:ligand interaction. The eukaryotic cell comprises a number of discrete membrane bound compartments, or organelles. The structure and function of each organelle is largely determined by its unique complement of constituent polypeptides. However, the vast majority of these polypeptides begin their synthesis in the cytoplasm. Thus organelle biogenesis and upkeep require that newly synthesized proteins can be accurately targeted to their appropriate compartment. This is often accomplished by amino-terminal signaling sequences, as well as post-translational modifications and secondary structure.

In one embodiment, the nucleic acid molecules expressing a mitochondrial targeting signal can encode amino acids comprising at least two, preferably 5-15, most preferably about 11 charged groups. In another embodiment, the targeting signal can contain a series of charged groups that cause the targeting signal to be transported into an organelle either against or down an electromagnetic potential gradient. Suitable charged groups are groups that are charged under intracellular conditions such as amino acids with charged functional groups, amino groups, nucleic acids, and the like. Mitochondrial localization/targeting signals generally consist of a leader sequence of highly positively charged amino acids. This allows the protein to be targeted to the highly negatively charged mitochondria. Unlike receptor:ligand approaches that rely upon stochastic Brownian motion for the ligand to approach the receptor, the mitochondrial localization signal of some embodiments is drawn to mitochondria because of charge.

In order to enter the mitochondria, a protein generally must interact with the mitochondrial import machinery, consisting of the Tim and Tom complexes (Translocase of the Inner/Outer Mitochondrial Membrane). With regard to the mitochondrial targeting signal, the positive charge draws the linked protein to the complexes and continues to draw the protein into the mitochondria. The Tim and Tom complexes allow the proteins to cross the membranes. Accordingly, one embodiment of the present disclosure delivers compositions of the present disclosure to the inner mitochondrial space utilizing a positively charged targeting signal and the mitochondrial import machinery. In another embodiment, PTD-linked polypeptides containing a mitochondrial localization signal do not seem to utilize the TOM/TIM complex for entry into the mitochondrial matrix, see Del Gaizo et al. (2003) Mol Genet Metab. 80(1-2):170-80.

### Modified Oligonucleotides

In another preferred embodiment, a nucleic acid molecule (e.g. vector) comprises a cytomegalovirus enhancer, a promoter, a human mitochondrial superoxide dismutase (SOD2) gene, an internal ribosome entry site (IRES) and a detectable marker gene. Preferably, the cytomegalovirus enhancer, the promoter, the human mitochondrial superoxide dismutase (SOD2) gene, the internal ribosome entry site (IRES) and the detectable marker gene are interposed between inverted terminal repeat sequences. The AAV vector comprises Rep, Cap, inverted terminal repeat (ITR) sequences and the AAV is selected from the group consisting of AAV-1 to AAV-9 serotypes.

In preferred embodiments, any one or more of the sequences comprise modified nucleobases. For example, certain preferred oligonucleotides of this invention are chimeric oligonucleotides. “Chimeric oligonucleotides” or “chimeras”, in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In one preferred embodiment, a chimeric oligonucleotide comprises at least one region modified to increase target binding affinity, and, usually, a region that acts as a substrate for RNAse H. Affinity of an oligonucleotide for its target (in this case, a nucleic acid encoding ras) is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target. In a more preferred embodiment, the region of the oligonucleotide which is modified to increase nutrient amino acid transporter mRNA binding affinity comprises at least one nucleotide modified at the 2′ position of the sugar, most preferably a 2′-O-alkyl, 2′-O-alkyl-O-alkyl or 2′-fluoro-modified nucleotide. Such modifications are routinely incorporated into oligonucleotides and these oligonucleotides have been shown to have a higher Tₘ (i.e., higher target binding affinity) than; 2′-deoxyoligonucleotides against a given target. The effect of such increased affinity is to greatly enhance RNAi oligonucleotide inhibition of nutrient amino acid transporter gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃ OCH₃, OCH₃O(CH₂)ₙCH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

In accordance with the invention, use of modifications such as the use of LNA monomers to enhance the potency, specificity and duration of action and broaden the routes of administration of oligonucleotides comprised of current chemistries such as MOE, ANA, FANA, PS etc (ref: Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

### Administration and Dosage

In a preferred embodiment of the method, the AAV-SOD2 compositions are administered via a systemic or mucosal route, or directly into a specific tissue, such as the liver, bone marrow, or into the tumor in the case of cancer therapy. Examples of systemic routes include, but are not limited to, intradermal, intramuscular, subcutaneous and intravenous administration. Examples of mucosal routes include, but are not limited to, intranasal, intravaginal, intrarectal, intratracheal and ophthalmic administration.

Treatment includes prophylaxis and therapy. Prophylaxis or therapy can be accomplished by a single direct administration at a single time point or multiple time points. Administration can also be delivered to a single or to multiple sites.

The subject (patient) can be any vertebrate, but will preferably be a mammal. Mammals include human, bovine, equine, canine, feline, porcine, and ovine animals. If a mammal, the subject will preferably be a human, but may also be a domestic livestock, laboratory subject or pet animal.

The compositions of the present invention preferably contain a physiologically acceptable carrier. While any suitable carrier known to those of ordinary skill in the art may be employed in the inventive compositions, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the compositions of this invention. Suitable biodegradable microspheres are disclosed, for example, in U.S. Pat. Nos. 4,897,268 and 5,075,109. The compositions of the present invention may also contain a substance designed to protect the antigen from rapid catabolism, such as aluminum hydroxide or mineral oil.

In general, the inventive compositions may be administered by injection (e.g., intraocular, intradermal, intramuscular, intravenous or subcutaneous), intranasally (e.g., by aspiration) or orally. In certain embodiments, the compositions of the present invention are in a form suitable for delivery to the mucosal surfaces of the airways leading to or within the lungs. For example, the composition may be suspended in a liquid formulation for delivery to a patient in an aerosol form or by means of a nebulizer device similar to those currently employed in the treatment of asthma.

The preferred frequency of administration and effective dosage will vary both from individual to individual, and with the known antigen against which an immune response is to be raised, and may parallel those currently being used in immunization with the known antigen. In general, the amount of polypeptide immunostimulant present in a dose (or produced in situ by the polynucleotide in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 ml to about 2 ml.

The word “about,” when used in this application with reference to the amount of active component in a dose, contemplates a variance of up to 5% from the stated amount.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Example 1

### Suppression of Mitochondrial Oxidative Stress Provides Longterm Neuroprotection in Experimental Optic Neuritis

Axonal loss is believed to contribute to persistence of visual loss in optic neuritis and MS. The mechanisms of injury are poorly understood. Here we investigated the contribution of mitochondrial oxidative stress and the effects of modulating mitochondrial antioxidant gene expression in the optic nerves of mice induced with EAE, with a focus on long-term neuroprotection.

Recombinant Adeno-Associated Virus (rAAV). The adeno-associated AAV vector backbone pTR-UF was used to accept the SOD2 and the RzSOD2 cDNAs. Gene expression was driven by the hybrid cytomegalovirus (CMV) and chicken β-actin promoter (SOD2 and RzSOD2). The resulting pTR-SOD2, and pTR-RzSOD2 plasmids were amplified, then purified and packaged as AAV serotype 2 vectors. The resultant rAAV-packaged SODs and humanized GFP control viruses were assayed and each virus preparation contained 10¹¹ to 10¹² genome copies per milliliter and 10⁹ to 10¹⁰ infectious center units per milliliter.

Cell Culture, immunochemical and RNA analysis of SOD2. Mouse fibroblasts (NIH/3T3) and retinal ganglion cells (RGC-5) were grown in Dulbecco's Modified Eagle Medium (DMEM) (Fisher Scientific) supplemented with 10% heat-inactivated fetal bovine serum and 1% penicillin streptomycin (Sigma) at 37° C. with 5% CO₂. Cells were grown in 15 cm dishes and were infected at multiplicities of infection (MOI) of 5,000 particles per cell. Two days after AAV infections, cells were harvested and mitochondria were isolated from AAV-SOD2 transfected cells and controls infected with AAV-GFP. Briefly this involved washing the trypsinized cells in cold PBS, followed by resuspension in a buffer consisting of 50 mM Tris-HCl, 0.21M D-mannitol, 70 mM sucrose, 0.1M PMSF, 3 mM CaCl₂, 20 mM EDTA, pH 7.5. Cells were then manually homogenized. The homogenates were centrifuged at 1200×g for 10 min at 4° C. The resulting supernatant containing the mitochondrial fraction was collected then centrifuged at 12000×g for 20 min at 4° C. The pellet containing the mitochondria was washed and resuspended in buffer consisting of 50 mM Tris-HCl, 10 mM EDTA, 20% sucrose pH 7.5, then stored at −80° C. for later analysis.

For immunodetection, 15 μg of protein from the isolated mitochondrial pellet was separated on a 10% SDS polyacrylamide gel and electro-transferred to a polyvinylidene fluoride membrane (BioRad). Protein content of the samples was measured using the BioRad Dc Protein Assay (BioRad, Hercules, Calif.). We immunostained the membrane with polyclonal anti-SOD2 antibodies (Stressgen Bioreagents, Victoria BC, Canada) and then goat anti-rabbit IgG horseradish peroxidase (HRP)-conjugated secondary antibodies (Sigma). We detected complexes using the enhanced chemiluminescence (ECL) system (Amersham Pharmacia Biotech, Piscataway, N.J.). The immunostained fragments were quantified by densitometry, using NIH Image (available by ftp from zippy.nimh.nih.gov/or from rsb.info.nih.gov/nih-image). Anti-mouse β-actin antibody was used as an internal control for protein loading. Each SOD2 signal was normalized to the β-actin signal from the same sample and the normalized values were expressed as a percentage of the signal from the control cells.

A superoxide dismutase assay kit (Calbiochem, San Diego, Calif.) was used to test SOD2 activity in the mitochondrial isolates according to the manufacturer's instruction. Briefly, the isolated mitochondria were incubated with 1-methyl-2-vinylpyridinium (R2) at 37° C. for 1 minute. The reagent 5, 6, 6a, 11b-tetrahydro-3,9,10-trihydroxybenzo[c]fluorine (R1) was then added. The R1 reagent undergoes alkaline autoxidation, which is accelerated by superoxide dismutase and yields a chromophore. The kinetic measurement of the 525 nm absorbance change was performed by Power Wave plate reader (Bio-TEK Instruments Inc., Winooski, Vt.) after the addition of R1. The SOD activity was determined from the ratio of the auto-oxidation rates measured in the samples and in the assay control, deionized water. One SOD-525 activity unit is defined as the activity that doubles the auto-oxidation rate of the assay control. SOD activity was expressed in SOD-525 units/mg protein.

To quantify SOD2 mRNA levels, total RNA of SOD2 transfected murine 3T3 cells was extracted with a kit (RNeasy Mini Kit; Qiagen, Valencia, Calif.), according to the manufacturer's specifications. For detection of transfected human SOD2 RNA we used a full length probe of human SOD2 cDNA. Mouse GAPDH DNA probe (905 bp/fragment) used as an internal control was purchased from Ambion. All probes used in northern blot analysis were labeled with α-³²P-ATP. Twenty micrograms of total RNA was fractionated in a 1.2% agarose gel containing 1% formaldehyde, transferred to a nylon membrane and fixed by UV cross-linking. The filter was hybridized at 68° C. for 1 hour with labeled probe in QuikHyb solution (Stratagene, Cat. No. 201220), washed with 2×SSC/0.05% SDS for 40 min at room temperature and then with 0.1×SSC/0.1% SDS for 40 min at 50° C. The probe was removed from the blot by incubating with 0.5% SDS in H₂O at 90° C. for 10 min. The filter was then re-equilibrated in the QuikHyb solution and reprobed with a new sequence. The hybridization signals were exposed on film overnight at −80° C. Radioactive signals were scanned and quantitated by using NIH image. Each SOD2 signal was normalized to the GAPDH signal from the same sample, and the normalized values were expressed as a percentage of the signal in the control.

Induction of EAE. All mice in this study were treated in accordance with the ARVO statement regarding the humane treatment of animals and with approval of the University of Florida Institutional Care and Use Committee. Experimental allergic encephalomyelitis was induced in mice by sensitization with 0.2 cc of sonicated homologous spinal cord emulsion in complete Freunds adjuvant (Difco, Detroit, Mich.) that was injected subdermally into the nuchal area two weeks after intraocular injection of rAAV or on the same day as the rAAV injections.

Intraocular Injections. Two microliters of rAAV (RzSOD2-20 mice, SOD2-50 mice) were injected into the vitreous cavity of DBA/1J mice. These mice were simultaneously sensitized for EAE. However, to allow sufficient time for expression of the AAV delivered transgene (SOD2) during early EAE, ten mice received intravitreal injections of AAV-SOD2 into the right eyes, but they were sensitized for EAE 2 weeks after intraocular injections and then euthanized 3 days later. As internal controls for the placebo effects of ocular injection, the left eyes of mice sensitized for EAE received AAV-GFP. Ten mice that received no intraocular injection served as controls for unadulterated EAE. Ten unsensitized animals served as normal controls, for comparison to the disease state. To confirm that MnSOD levels were increased by AAV-SOD2 or decreased by AAV expressing the anti-SOD2 ribozyme, the right eyes of 10 additional mice were similarly injected with AAV-SOD2 and an additional 10 mice injected with AAV-RzSOD2. The left eyes of these 20 mice were inoculated with AAV-GFP. Analysis of mitochondrial SOD was performed on the pooled right optic nerves of AAV-SOD2 and AAV-RzSOD2 inoculated mice relative to the pooled left eyes that were treated with AAV-GFP and excised after one month for immunoblotting with the MnSOD antibody as described above for cultured cells.

Detection of ROS and Mitochondrial Selective Probes. Mice received an overdose of sodium pentobarbital, 3 days (10 mice) and 6 days (10 mice) after antigenic sensitization. The globes and attached optic nerves were immediately dissected out then processed for fluorescent double staining for comparisons to ocular specimens obtained from normal animals (10 mice) not sensitized for EAE. To detect intracellular ROS generation, we used two probes (Molecular Probes, Eugene, Oreg.). The probe 2′-7′ dichlorofluorescein diacetate (DCFDA) was used to detect hydrogen peroxide. DCFDA has no fluorescence until it passively diffuses into cells where intracellular esterase cleaves the acetates, and the oxidation of DCFDA by H₂O₂ produces a green-fluorescent signal. Dihydroethidium (DHE) was used to detect intracellular superoxide (⁻O). Superoxide oxidizes DHE to a red-fluorescent signal. MitoTracker dyes (Molecular Probes, Eugene, Oreg.) (red, M-7512 or green, M-7514) were co-stained with ROS probes. After a brief rinse in PBS, tissues were incubated for 20 minutes at 37° C. with a mixture of 10 μM DCFDA plus 0.3 μM MitoTracker Red or 2.5 μM dihydroethidium plus 0.1 μM MitoTracker Green. Tissues were washed with PBS, fixed with cold 4% paraformaldehyde for 2 hours, processed for cryomicroscopy then observed under a fluorescence microscope (Leitz) or confocal microscope (Biorad).

Immunohistochemistry. The retinas and optic nerves were immediately dissected out of 10 mice three days after antigenic sensitization for EAE, and 10 mice six days after EAE sensitization, for comparison to 10 control mice three days after inoculation with only Freunds adjuvant and 10 normal unsensitized mice. Following washes in increasing concentrations of sucrose PBS buffer, the isolated tissues were snap frozen and stored at −20° C. Tissues were sectioned on a cryostat (Leitz). After blocking in 5% normal goat serum, they were then reacted with primary anti-macrophage (epitope: F4/80 antigen) or anti-oligodendrocyte (epitope: full length cyclic nucleotide phosphodiesterase (CNPase)) antibodies (Abcam, Inc., Cambridge, Mass.) followed by incubation with Cy2 conjugated anti-mouse secondary antibodies (Jackson Laboratories) then visualized by fluorescence microscopy. Additionally, apoptotic cell death was assessed with a terminal deoxynucleotidyltransferase-mediated dUTP nick end labeling (TUNEL) reaction kit, according to the manufacturer's specifications (Roche, Indianapolis, Ind.).

GFP Expression. The retinas and optic nerves of GFP infected eyes were sectioned and examined for expression of GFP one month after sensitization for EAE. To determine whether inflammatory cells or oligodendrocytes expressed GFP, we reacted the sectioned tissues against the anti-macrophage or anti-oligodendrocyte antibody that were counterstained red with cy3. The tissues were then examined by fluorescence microscopy for co-localization or GFP with the cellular markers.

MRI Analysis. Two weeks, 1, 3, 5, 6 and 12 months after EAE sensitization and viral inoculations, high-resolution 3D MRI of mouse optic nerve was performed using a 4.7 Tesla magnet (Oxford). The animals were anesthetized with IsoFlo (isoflurane 1.5-2%), in a prone position with their heads firmly fixed in a purpose-built surface coil. T1 weighted 3D image acquisitions were performed immediately following intraperitoneal administration of gadolinium (Gd)-DTPA (Berlex Lab) at a dose of 0.2 mmol/kg of body weight. Using the Silicon Graphics O2 workstation, ParaVision 2.212 and software developed by the UF Brain Institute MRI core, the volume of the optic nerve was quantified. For statistical analysis, the AAV-GFP inoculated left eyes were compared to the right eyes that received the AAV-SOD2 rescue gene. Statistical analysis was performed by Student's t-test for unpaired data.

Light and Electron Microscopy. Mice received an overdose of sodium pentobarbital 1, 3 and 12 months after viral inoculation. They were then immediately perfused intracardially with fixative consisting of 4% paraformaldehyde and 2% glutaraldehyde in 0.1 M PBS buffer (pH 7.4). For detection of in vivo H₂O₂, with a mixture consisting of 2 mM cerium chloride, 10 mM 3-amino-1,2,4-triazole, 0.8 mM NADH, 0.1 M PBS buffer (pH 7.5), and 7% sucrose followed by perfusion with the fixative. The eyes with attached optic nerves were dissected and further processed by immersion in 2.5% glutaraldehyde and then postfixed in 1% osmium tetroxide, 0.1 M sodium cacodylate-HCl buffer (pH 7.4). Tissue was then dehydrated through an ethanol series to propylene oxide, infiltrated, and embedded in epoxy resin that was polymerized at 60° C. overnight. Semi-thin longitudinal sections (0.5-1 μm) of the optic nerve head and retrobulbar nerve were made and stained with toluidine blue.

Ultrathin sections (90 nm) were cut and placed on nickel grids for immunocytochemistry. Nonspecific binding of antibodies was blocked by floating the grids on 5% normal goat serum in 0.01 M Tris-buffered saline, (pH 7.2) with Tween 20 (TBST) for 30 min. They were then reacted with rabbit anti-MnSOD antibodies. After washes in 0.1 M PBS, the grids were reacted with the secondary goat anti-rabbit IgG antibodies conjugated to 10 nm gold for 1 hr at room temperature. After washes in buffer, grids were rinsed in deionized water. For examination by low magnification transmission electron microscopy, the immunogold particles were enlarged by silver enhancement using a kit (Ted Pella, Redding, Pa.) according to the manufacturer's specifications. To check for nonspecific binding of the secondary antibody, control grids were incubated in the buffer, followed by the gold-labeled antibody. Immunolabeled and control specimens were photographed by transmission electron microscopy with and without post-staining (model; H-7000 or H7600; Hitachi, Tokyo, Japan) operating at 75-80 kV.

Morphometric Analysis: Morphometric analysis was performed in masked fashion as previously described. Briefly, images of toluidine blue-stained sections of the retina and optic nerve were captured with a video camera mounted on a light microscope, and then the data were entered into the computer memory. Myelin fiber areas and ganglion cell counts were quantified using the NIH Image software. For statistical analysis, rAAV-SOD2 and RzSOD2 inoculated right eyes were compared with the left eyes that received the control virus rAAV-GFP, as well as to the uninoculated eyes of animals with EAE and the eyes of normal unsensitized animals. Statistical analysis was performed by Student's t-test for unpaired data.

### Results:

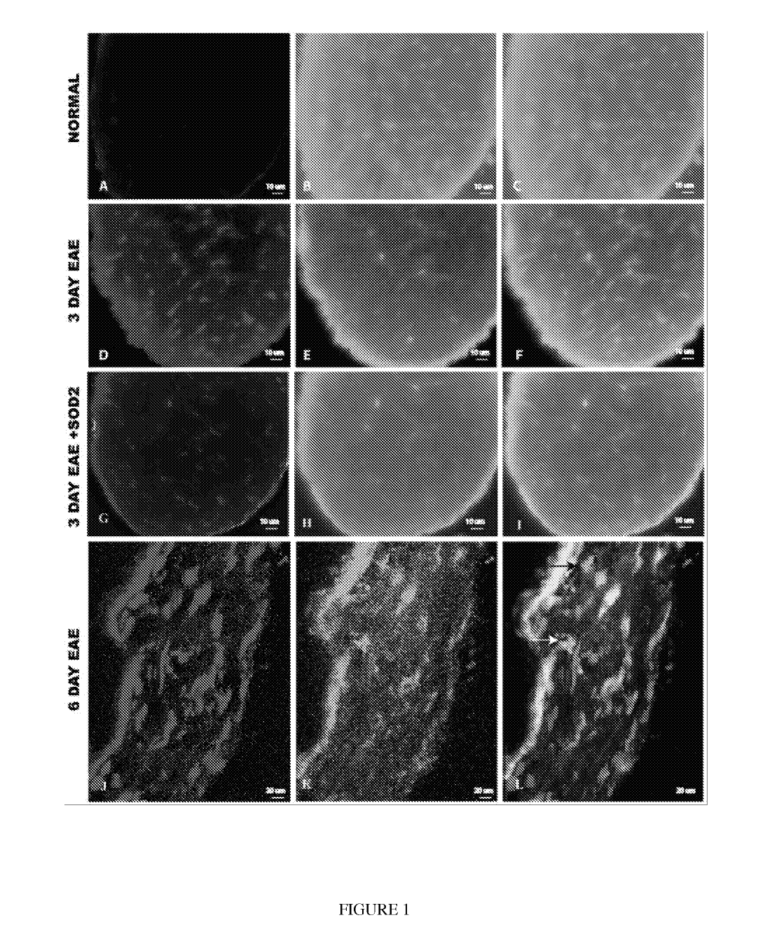

Mitochondrial oxidative stress starts early. As an initial gauge of ROS activity, we used the fluorescent probes DCFDA and DHE. DCFDA detects H₂O₂ while DHE is a probe for O₂⁻. The optic nerves of 10 mice euthanized 3 days after antigenic sensitization and 10 mice euthanized 6 days after antigenic sensitization as well as 10 normal unsensitized mice were excised and incubated in DCFDA and MitoTracker Red or dihydroethidium and MitoTracker Green. Fluorescence microscopy of the cyrosectioned EAE specimens revealed that relative to normal optic nerve in which superoxide anion was undetectable (FIGS. 1A and 1C), superoxide detected by red labeling with dihydroethidium was seen as early as 3 days after sensitization for EAE (FIG. 1D). ROS activation was associated with some loss of optic nerve mitochondrial membrane potential as detected by diminished MitoTracker Green labeling (FIG. 1E) relative to the normal optic nerve (FIG. 1B). Due to the loss of membrane potential, we could not use co-localization shown in the merged panel (FIG. 1F) to prove that mitochondria were the source of superoxide anion in early EAE.

We also found that MitoTracker Red labeling of mitochondria (FIG. 1J) was associated with hydrogen peroxide detected by green labeling with 2′-7′ dichlorofluorescein diacetate (DCFDA) (FIG. 1K). Co-localization with MitoTracker suggested the source of hydrogen peroxide was mitochondria of the optic nerve (FIG. 1L). In general, loss of membrane potential did not appear to be associated with the presence of the hydrogen peroxide. However, loss of MitoTracker Red labeling indicated that mitochondrial membrane potential was diminished at several perivascular foci where hydrogen peroxide was highly expressed (FIG. 1L). DCFDA labeling was absent in the optic nerves of unsensitized control mice.

We examined the effect of SOD2 gene inoculation on dismutation of superoxide during early EAE. Ten mice received intravitreal injections of AAV-SOD2 into the right eyes. They were sensitized for EAE two weeks after intraocular injections to allow sufficient time for expression of the AAV delivered transgene (SOD2), then euthanized 3 days after antigenic sensitization. Examination of SOD2 inoculated optic nerves revealed diminished DHE (FIG. 1G) and MitoTracker Green labeling (FIG. 1H-I), relative to eyes not treated with AAV-SOD2. Clearly, SOD2 gene inoculation reduced accumulation of superoxide anion.

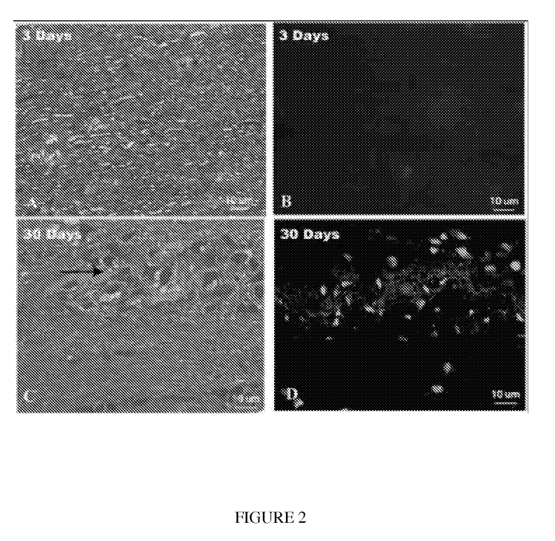

Inflammatory cells are absent during early EAE. We excluded inflammatory cells as the source of superoxide anion and hydrogen peroxide in early EAE by light microscopic examination of toluidine blue stained optic nerves (FIG. 2A) and by immunofluorescence labeling with an anti-macrophage antibody (FIG. 2B). As positive controls the presence of inflammatory cells is shown in the one month EAE optic nerve (FIG. 2C-D). Clearly, ROS activation began prior to the CNS infiltration by inflammatory cells, long thought to be the source of mediators of tissue injury in EAE and MS.

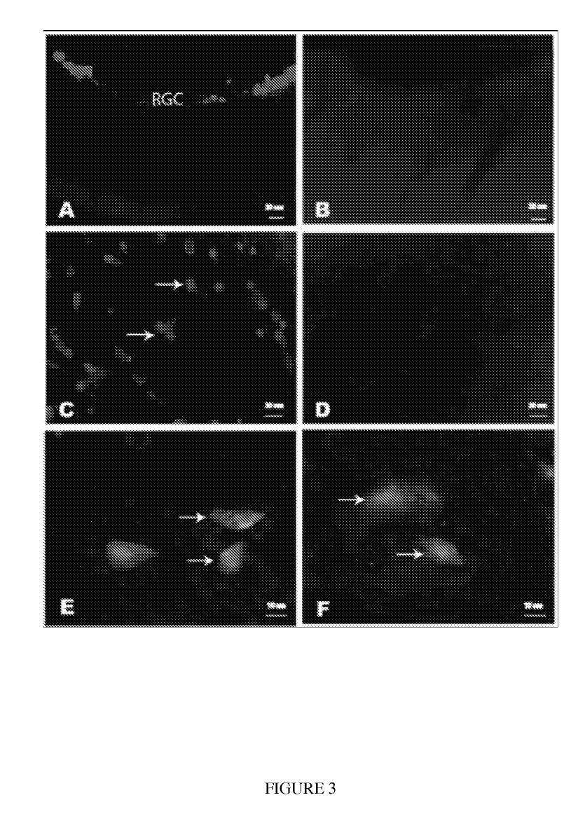

Apoptosis starts during early EAE. Since ROS exposure has been linked to loss of mitochondrial membrane potential, leading to release of cytochrome c and apoptosis, we examined the optic nerves and retinas of 10 mice sensitized for EAE 3 days earlier for apoptosis. Ten mice inoculated with only Freunds adjuvant served as controls. We found TUNEL positive cells in the ganglion cell layer of the retina in three-day EAE animals (FIG. 3A), but not in controls inoculated with the adjuvant (FIG. 3B). In addition, we saw TUNEL positive cells in the three-day EAE optic nerve (FIG. 3C), but not in controls inoculated with the adjuvant (FIG. 3D). The TUNEL positive cells in the nerve were identified as oligodendrocytes by co-localization with an anti-oligodendrocyte antibody (FIG. 3E). Oligodendrocytes in the adjuvant inoculated nerves were TUNEL negative (FIG. 3F).

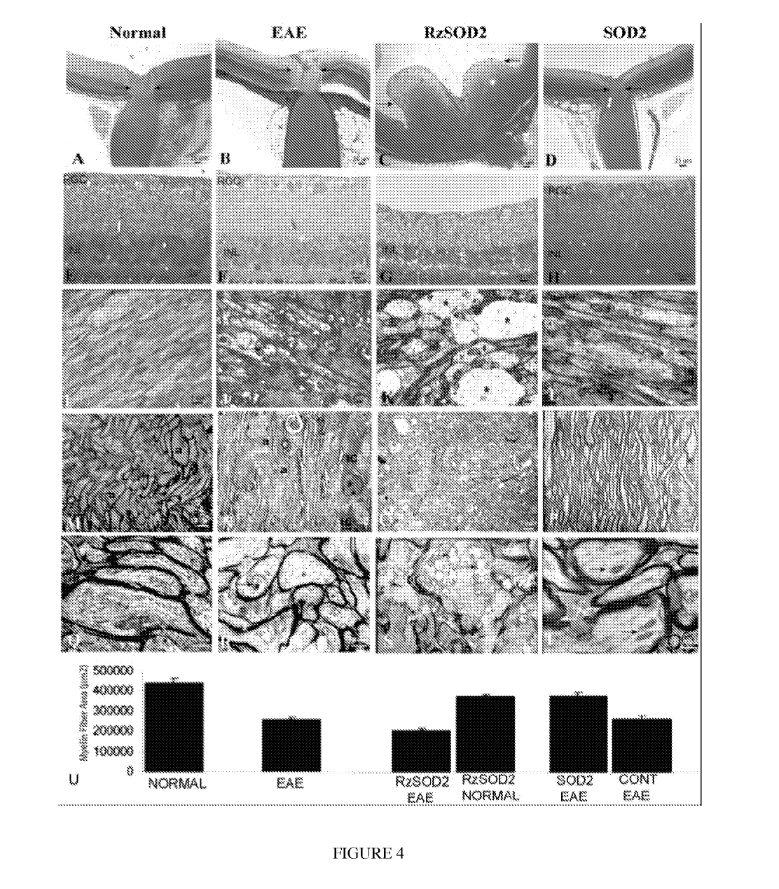

Acute experimental optic neuritis. One month after sensitization of 10 mice for EAE, we found electron-dense cerium perhydroxide reaction product formed by the reaction of perfused cerium chloride and endogenous hydrogen peroxide within mitochondria (FIG. 4R), some of which were swollen and showed dissolution of cristae (FIG. 4J). These mitochondrial findings were not limited to fibers that had lost their myelin sheaths, often considered the hallmark of MS and EAE, but rather were more widespread. Myelinated axons also contained swollen mitochondria that exhibited disorganization and dissolution of cristae, some to the point that only a double membrane sheath identified the organelle. At this stage of EAE, mononuclear inflammatory cells involved in active demyelination were prevalent in the retrobulbar nerve (FIG. 4N). Quantitative comparisons with the optic nerves of 5 normal mice that were not sensitized for EAE showed that eyes from animals undergoing acute EAE lost half of their optic nerve myelin fiber area relative to the normal optic nerve (FIG. 4U).

Lowering mitochondrial antioxidant protection exacerbates acute optic neuritis. To support the pathogenicity of mitochondrial ROS activity in EAE, we first suppressed antioxidant defenses in the organelle by using a ribozyme designed to target the SOD2 mRNA for destruction. This ribozyme, delivered by an AAV-2 vector was injected into the vitreous cavity of the right eyes of 10 mice, while the left eyes were injected with AAV-GFP. One month later, immunoblots revealed MnSOD level was reduced by half in the pooled optic nerves of AAV-RzSOD2 injected eyes relative to the left eyes injected with AAV-GFP. Next, we tested the effect of lowered SOD2 level on experimental optic neuritis by injecting the AAV containing anti-SOD2 ribozyme into the right eyes of 10 animals sensitized for EAE and 10 normal mice that were not sensitized for EAE. The left eyes of both groups received control treatment by injection with AAV-GFP. Mice were euthanized a month later. We found that reducing mitochondrial SOD2 activity with the anti-SOD2 ribozyme exacerbated mitochondrial and axonal swelling, RGC and myelin fiber loss (FIGS. 4C, 4G, 4K, 4O, 4S and 4U) relative to controls, normal mice that were injected with the AAV-RzSOD2 but not sensitized for EAE and those that were sensitized for EAE but treated by inoculation with AAV-GFP (FIG. 4U). In fact, the severe optic nerve head swelling induced by the ant-SOD2 ribozyme accompanied by EAE shown in FIG. 4C was worse than any other animal examined in this study. Relative to AAV-RzSOD2 treated EAE eyes, the EAE only optic nerve head (FIGS. 4B and 4J), retina (FIG. 4F) and retrobulbar nerve (FIGS. 4N and 4R) showed less severe RGC, axonal and mitochondrial swelling and demyelination. The normal optic nerve head (FIGS. 4A and 4I), retrobulbar nerve (FIGS. 4M and 4Q) and retina (FIG. 4E) are shown for comparison to untreated EAE and ribozyme treatment (FIG. 4U).

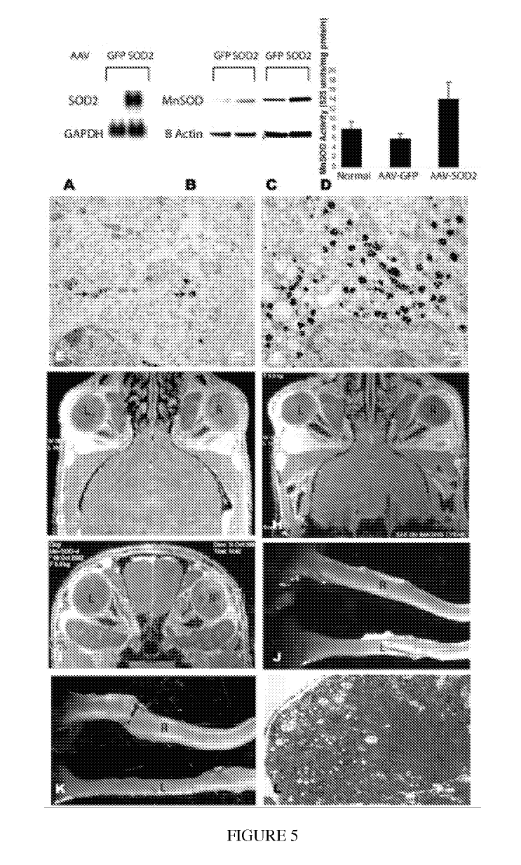

Increasing mitochondrial defenses ameliorates acute optic neuritis. Next, we bolstered mitochondrial anti-ROS defenses. In vitro, inoculation of murine fibroblasts (3T3) with the human SOD2 AAV resulted in expression of human SOD2 mRNA (FIG. 5A), increased mitochondrial MnSOD protein approximately 6-fold in rat retinal ganglion cells (RGC-5) (FIG. 5B) and almost 2-fold in the murine optic nerve (FIG. 5C). Mitochondrial SOD activity in cultured retinal ganglion cells (RGC-5) was increased approximately 2.5-fold relative to cells treated with AAV-GFP (FIG. 5D). Injection of AAV containing the SOD2 gene into the mouse eye increased MnSOD immunogold in the optic nerve (FIG. 5F) relative to control, AAV-GFP inoculation (FIG. 5E). The MnSOD immunogold was found predominantly in the mitochondria of axons of the optic nerve.

We compared the AAV-SOD2 inoculated right eyes of 10 mice sensitized for EAE and euthanized a month later to the left eyes that received control treatment by injection with AAV-GFP. We found that AAV-SOD2 inoculated nerves (FIGS. 4D, 4H, 4L, 4P, 4T and 4U) exhibited less mitochondrial and axonal swelling, with 46% more myelin fiber preservation than the contralateral nerves treated by inoculation with AAV-GFP (FIG. 4U). Suppression of mitochondrial injury by SOD2 was not limited to demyelinated axons, but also seen in myelinated axons that exhibited less hydropic degeneration, disorganization and dissolution of cristae (FIG. 4T).

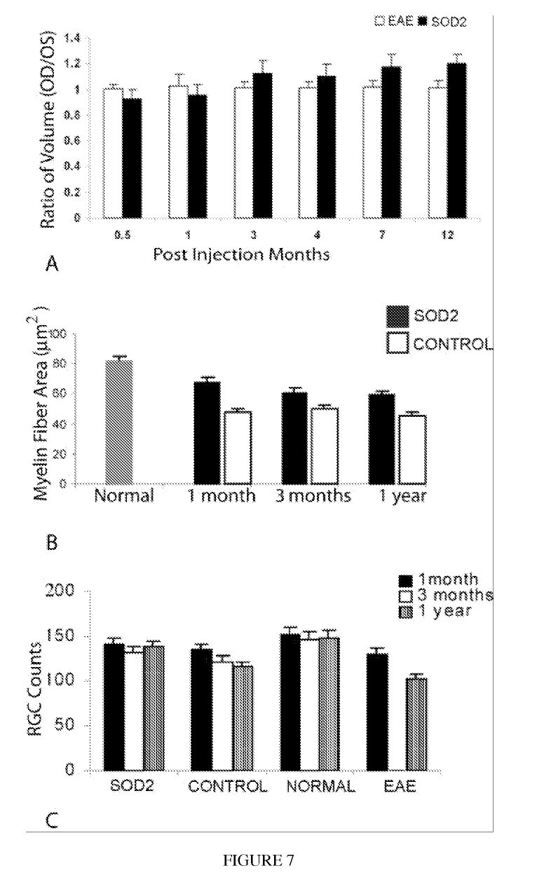

Long-term antioxidant gene transfer suppresses optic neuritis. Next, we tested the effects of the SOD2 construct on chronic EAE, focusing on long-term neuroprotection. The right eyes of 20 mice received intraocular injections of AAV-SOD2 and the left eyes were injected with a control AAV expressing GFP. To follow the effects of modulation of experimental optic neuritis in living animals we used volume measurements of the optic nerve obtained by serial 3-D Magnetic Resonance Imaging (MRI). MRI of our animals was performed at 2 weeks, then at 1, 3, 4, 6, 7 and 12 months following sensitization for EAE. Because ocular injections may be associated with the release of growth factors that can sometimes offer a protective effect, another group of 10 animals that received no ocular viral inoculation was sensitized for EAE and evaluated by MRI. Optic nerve volumes between the right and left eyes of this group were the same throughout the course of EAE [ratio OD/OS=1] (FIG. 7A). Though disease activity is somewhat variable between animals with EAE, the severity of optic neuritis did not substantially vary between eyes of the same animal.

Two and four weeks after EAE sensitization, optic nerve volumes for the control nerves (infected with AAV-GFP) increased relative to SOD2 treatment (FIGS. 5G and 7A), reflecting an initial decrease in swelling with SOD2 treatment. By the third month and thereafter, we detected a loss of optic nerve volume that was suppressed by SOD2 gene inoculation as long as 1 year after sensitization for EAE, the longest interval studied (FIGS. 5H, 5I and 7A). This observation suggested to us that mitochondrially targeted anti-ROS gene transfer suppressed optic nerve degeneration. As further proof of this observation, we next confirmed our serial in vivo MRI findings using histopathology as the gold standard.

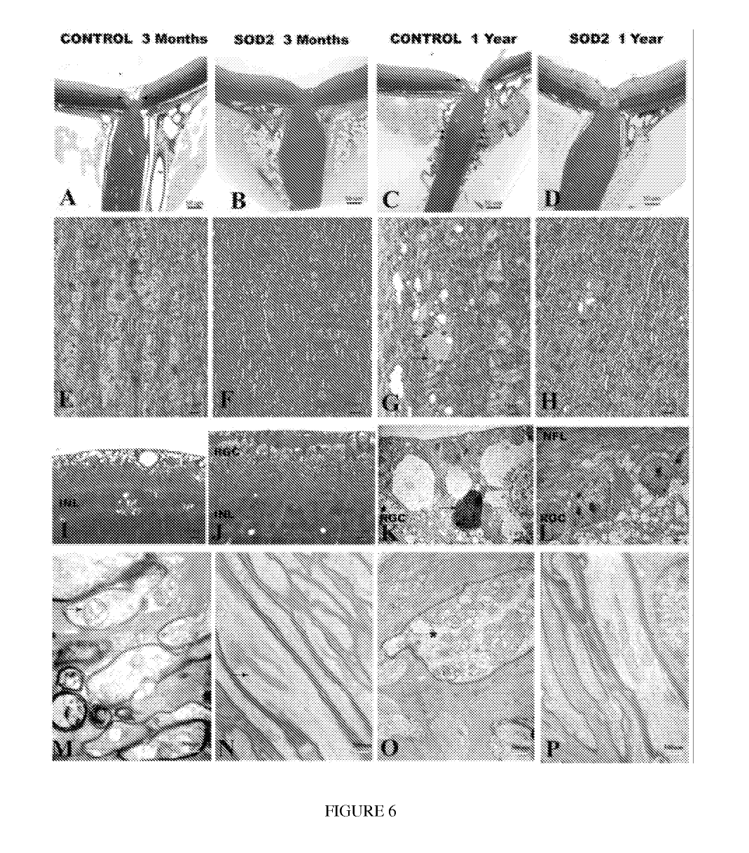

Half of the 20 mice that received intraocular injections of AAV-SOD2 were euthanized 3 months after EAE sensitization and the other 10 mice at 1 year. Postmortem examinations confirmed the long-term protective effect of SOD2. Excised optic nerves clearly show that, relative to treatment by AAV-GFP inoculation, the optic atrophy characteristic of EAE was suppressed by SOD2 gene inoculation, 3 months (FIG. 5J) and 1 year after antigenic sensitization (FIG. 5K). The demyelination of EAE was also evident in the spinal cord (FIG. 5L) and untreated optic nerve (FIGS. 6-7), where myelin fiber area dropped by 49% relative to normal animals after 1 year (FIG. 7B). With SOD2, myelin fiber area diminished, but only by 23%. Thus, mitochondrial SOD offered a 2-fold protective effect when compared with the normal optic nerve. SOD2 infected nerves had 51% more myelin fiber preservation relative to the untreated EAE optic nerve, although somewhat less when measured against the eyes injected with AAV-GFP (31%). Excavation of the optic nerve head, retrobulbar nerve atrophy (FIGS. 6A and 6C) and myelin fiber loss (FIGS. 6E and 6G) were suppressed with SOD2 (FIGS. 6B, 6D, 6F and 6H). Degenerating axons, some with aggregation of mitochondria, hydropic degeneration and loss of cristae provided evidence of ongoing neurodegeneration not only at 3 months (FIG. 6M), but also at 1 year after sensitization for EAE (FIG. 6O). These changes were suppressed by SOD2 (FIGS. 6N and 6P).

Antioxidant gene transfer suppresses neuronal degeneration. The protective effect of SOD2 seen in the optic nerve was mirrored in the retina, the site of retinal ganglion cell bodies. Unlike our observations in acute EAE where substantial degeneration of the nerve fiber layer and loss of RGCs was mainly evident in eyes inoculated with anti-SOD2 ribozyme, RGC loss predominated in the chronic stages of EAE (FIGS. 6I and 6K). AAV-SOD2 treatment helped to preserve the nerve fiber layer and RGCs (FIGS. 6J and 6L). Quantitative analysis revealed a 32% loss of RGCs a year after sensitization for EAE, relative to normal animals (FIG. 7C). We found that SOD2 suppressed ganglion cell loss 4-fold, limiting it to 7% in EAE. Even at 1 year, RGCs of unprotected and AAV-GFP inoculated EAE animals were still undergoing apoptosis (FIG. 6K). This suggests that the neurodegenerative process was ongoing and active even this late in the disease course.

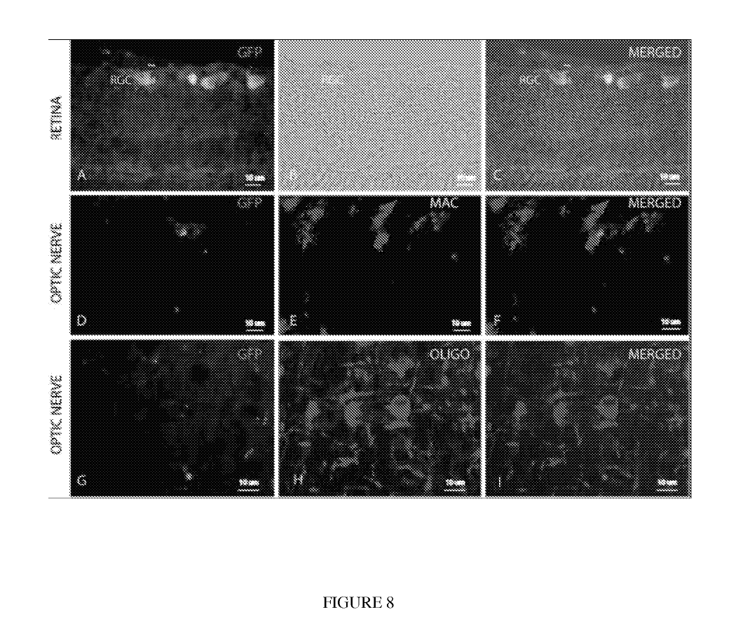

GFP expression in EAE: One month after sensitization for EAE, we found GFP expression in the retina exclusively in ganglion cells (FIGS. 8A-8C). While some GFP labeling was evident in the optic nerve (FIGS. 8D and 8G), it did not appear to co-localize with either inflammatory cells (FIGS. 8E-8F) or oligodendrocytes (FIGS. 8H-8I). Thus, the tropism of AAV2 for RGCs was not altered by EAE, suggesting that the protective effect of SOD2 was likely due to expression of this antioxidant enzyme in mitochondria of RGCs and their axons (FIGS. 5C and 5F).

Discussion: We have provided evidence showing that the pathway leading towards neurodegeneration in the EAE animal model of MS can be ameriorated by suppression of mitochondrial oxidative stress that began prior to the infiltration of inflammatory cells classically believed to be the mediators of disease activity. While demyelination is the classic target of disease activity in EAE and MS, axonal and neuronal loss is becoming increasingly recognized as the major cause of persistent clinical disability with mitochondria playing a substantial role in the neurodegenerative process. Loss of mitochondrial membrane potential can increase the release of cytochrome c, one pathway leading to neuronal apoptosis that is mediated by the Bcl-2 family of proteins Bcl-2 increases in MS lesions further support a role for apoptosis in the axonal and neuronal degeneration of MS. We showed that increasing mitochondrial defenses against superoxide suppressed loss of mitochondrial membrane potential and protected RGCs and axons of the optic nerve. Our previous work focused on ROS released by inflammatory cells, clearly they were not the source of superoxide we found in early pre-inflammatory EAE.

As we have found here in the EAE optic nerve, a superoxide burst from mitochondria has been described with RGC injury. Generation of mitochondrial superoxide is predominantly mediated by complex I and III of the electron transport chain. Complex I deficiency in longstanding MS has been attributed to oxidative stress. Loss of oxidative phosphorylation activity in MS has been detected that are comparable to or exceed the levels found in disorders of optic nerve degeneration associated with mutated mtDNA. Tajouri and co-workers also found that the expression of mitochondrial ATP synthase and cytochrome b was altered, thus implying deficits in oxidative phosphorylation induced by oxidative stress may contribute to axonal and mitochondrial injury in MS. While superoxide may mediate injury directly, peroxynitrite formed by the reaction of superoxide and nitric oxide, mediated nitration of key mitochondrial proteins in vitro and in the EAE nervous system perhaps contributing to deficits in oxidative phosphorylation.

Our previous work had detected endogenous increases in expression of MnSOD in mitochondria, induced by cytokines released by infiltrating inflammatory cells, in the unadulterated optic nerves of EAE animals. However, this endogenous increase of MnSOD was predominantly limited to inflammatory cells, microglial and astroglial cells, but did not appear to occur in axons or oligodendroglia indigenous to the optic nerve where it could have produced a neuroprotective effect. The relatively lower levels of mitochondrial SOD in oligodendroglial cells and axons that we previously detected likely increased their vulnerability to the effects of oxidative stress. Here, by increasing MnSOD expression, we were able to suppress not only myelin loss of the optic nerve, but also mitochondrial vacuolization, swelling and dissolution of cristae of optic nerve axons. The localization of the GFP reporter gene to retinal ganglion cells and increased MnSOD in axons of the optic nerve suggests that this protective effect was due predominantly to the neuroprotection of neurons and axons in EAE.

Suppression of axonal damage by AAV-SOD2 was apparent not only at foci of demyelination, but also in the optic nerve head, which does not contain myelinated axons. At these sites, axons with normal appearing myelin sheaths and unmyelinated axons of the ONH exhibited mitochondrial swelling with dissolution or disorganization of cristae. These abnormalities were substantially ameliorated by genetically increasing mitochondrial SOD2, thus protecting axons and myelin in the optic nerve. On the other hand, increasing oxidative stress, by reducing SOD2 gene expression with the ribozyme, increased our findings of cystic mitochondria, devoid of stainable contents and cristae. This is strong evidence supporting the role that reduction of mitochondrial oxidative stress can play in modulating experimental optic neuritis.

While inflammatory cells transect axons and cause neurodegeneration in MS, we showed here that mitochondrial ROS suppression with AAV-SOD2 was effective not only before and during the inflammation, but beyond after the inflammation subsided. This is not unlike MS, which eventually becomes a disease characterized by progressive neuronal and axonal degeneration. In EAE we found that apoptosis of RGCs together with mitochondrial and axonal degeneration were still active one year after sensitization for EAE, long after the inflammatory phase had subsided. Increasing mitochondrial antioxidant defenses provided long-term neuroprotection against RGC and axonal loss.

At the present time treatment for optic neuritis patients that do not recover vision is somewhat limited. Recent OCT measurements showing loss of macular volume in MS and optic neuritis patients suggests this may be due to loss of RGCs. Fortunately, visual function tests for most patients, followed for ten years after an initial attack of optic neuritis, show relatively mild impairment. However, for those with severe visual loss that has persisted for more than six months there is no remedy. Intravenous immunoglobulin proved unsuccessful in promoting restoration of visual function in multiple sclerosis patients blinded by recurrent attacks of optic neuritis. Because the neurodegeneration of MS does not appear to be substantially driven by inflammation, it is somewhat refractory to immunomodulatory drugs. Although these drugs suppress the inflammatory phase of the disease, an additional approach is needed to tackle the neurodegenerative component.

We have shown here that mitigation of ganglion cell death and loss of axons and myelin in experimental optic neuritis may be achieved by genetically induced expansion of mitochondrial defenses against superoxide. Increasing antioxidant defenses in the optic nerve using the AAV viral vector offers some promise for the future. The optic nerve is a readily accessible site for gene transfer, particularly with the AAV2 vector that selectively infects RGCs. The optic nerve is rich in mitochondria, which are widely accepted as the major intracellular source of ROS, thus making the nerve more susceptible to mitochondrial perturbations. In addition, RGCs whose axons comprise the optic nerve are highly dependent on oxidative phosphorylation and the optic nerve is also a frequent and initial site of involvement in MS. Increasing mitochondrial SOD expression provided long-term neuroprotection against EAE in the optic nerve for most of the lifespan of a laboratory mouse. Still, the injury that we detected in early EAE suggests that this approach may have the best chances of success if initiated at the earliest stages of disease, in order to reduce the cumulative injury beyond which loss of function becomes irreversible. Whether a similar strategy applied to patients may help avert the demise of axons, neurons and oligodendroglia in optic neuritis and MS remains to be demonstrated.

### Example 2

### Mitochondrial Protein Nitration Primes Neurodegeneration in Experimental Autoimmune Encephalomyelitis

To determine whether mitochondrial dysfunction plays an important role in the neurodegeneration of the EAE animal model of MS and that this process begins much earlier than currently believed.

Induction and Scoring of EAE: Experimental autoimmune encephalomyelitis was induced in 56 DBA/1J mice by sensitization with 0.2 ml of sonicated homologous spinal cord emulsion in complete Freund's adjuvant (Difco) injected subdermally into the nuchal area. Control animals (26 mice) received subdermal inoculation with Freund's adjuvant, and 30 mice were used as normal controls. Paralysis was graded on a scale of 0-5 with increasing severity of disease. Mice were humanely cared for in a veterinarian-supervised animal care facility that is fully accredited by the American Association of Laboratory Animal Science. At the prescribed interval they were euthanized by an overdose of sodium pentobarbital.

Mitochondrial Isolation and Immunodetection of ROS: Mitochondrial proteins were isolated from the optic nerves, retinas, brains, and spinal cords of 20 normal mice, 20 mice euthanized 3 days after sensitization for EAE, and 20 mice euthanized 6 days after antigenic sensitization. For comparisons to EAE, 20 normal unsensitized animals and 20 mice inoculated with Freund's adjuvant only and euthanized 3 days later served as controls. Mitochondria were isolated from excised CNS tissues (Fernandez-Vizarra, et al. (2002) Methods 26, 292-297). Briefly, this involved washing tissues in cold PBS, followed by resuspension in a buffer consisting of 50 mM Tris-HCl, 0.21 M D-mannitol, 70 mM sucrose, 0.1 M phenylmethylsulfonyl fluoride, 3 mM CaCl₂, 20 mM EDTA, pH 7.5. Tissues were then manually homogenized. The homogenates were centrifuged at 1200 g for 10 min at 4° C. The resulting supernatant containing the mitochondrial fraction was collected and then centrifuged at 12,000 g for 20 min at 4° C. The pellet containing the mitochondria was washed and resuspended in buffer consisting of 50 mM Tris-HCl, 10 mM EDTA, 20% sucrose, pH 7.5, and then stored at −80° C. for later analysis.

For immunodetection, 15 μg of protein of the isolated mitochondrial pellet or cytoplasmic supernatant were separated on a 10% SDS-polyacrylamide gel and electro-transferred to a polyvinylidene fluoride membrane (Bio-Rad). For detection of oxidative stress, we immunostained the membrane with mouse monoclonal nitrotyrosine or inducible nitric-oxide synthase (iNOS) antibodies (Abcam, Cambridge, Mass.). For normalization of sample loading, we used a mitochondrial loading control VDAC1/Porin antibody (Abcam, Inc., Cambridge, Mass.). Goat anti-mouse IgG or goat anti-rabbit IgG horseradish peroxidase-conjugated secondary antibodies (Sigma) were reacted against the respective primary antibody. We detected complexes using the enhanced chemiluminescence (ECL) system (GE Healthcare).

Identification of Nitrated Mitochondrial Proteins-We excised the nitrated protein bands and digested proteins in the excised bands with trypsin in situ. The resulting peptides were extracted and analyzed by mass spectrometry by the University of Florida Biotechnology Core Laboratory. In brief, capillary reverse phase HPLC separation of protein digests was performed on a 10-cm×75-μm inner diameter PepMap C18 column (LC Packings, San Francisco, Calif.) in combination with a home-built capillary HPLC system operated at a flow rate of 200 nl/min. In-line mass spectrometric analysis of the column eluate was accomplished by a quadrupole ion trap instrument (LCQ; ThermoFinnigan, San Jose, Calif.) equipped with a nanoelectrospray source. Fragment ion data generated by data-dependent acquisition via the LCQ were searched against the NCBI nr sequence data base using the SEQUEST (ThermoFinnigan) and Mascot (Matrix Science, Boston) data base search engines. In general, the score for SEQUEST protein identification was considered significant when dCn was equal to 0.08 or greater and the cross-correlation score was greater than 2.2. MASCOT probability-based MOWSE scores above the default significant value were considered for protein identification in addition to validation by manual interpretation of the tandem mass spectrometry data.

Immunohistochemistry: The retinas, optic nerves, spinal cords, and brains were immediately dissected out of 10 mice 3 days after antigenic sensitization for EAE, and 10 mice 6 days after EAE sensitization, for comparisons to 10 control mice 3 days after inoculation with only Freund's adjuvant and 10 normal unsensitized mice. Following washes in increasing concentrations of sucrose PBS buffer, the isolated tissues were snap frozen and stored at −20° C. Tissues were sectioned on a cryostat. After blocking in 5% normal goat serum for 30 min, they were then reacted with primary nitrotyrosine, iNOS, antimacrophage, or a mouse monoclonal antibody directed against cyclic nucleotide phosphodiesterase (Abcam, Inc., Cambridge, Mass.) as an oligodendrocyte marker. For detection of inflammatory cells, we used a pan-macrophage antibody (Abcam, Inc., Cambridge, Mass.). After an overnight incubation at 4° C., the specimens were washed in PBS followed by an overnight incubation with Cy2- or Cy3-conjugated anti-mouse secondary antibodies (Jackson ImmunoResearch). After washes, specimens were visualized by fluorescence microscopy. Quantitative analysis of iNOS induced fluorescence in EAE, and control optic nerve, retina, brain and spinal cord specimens were obtained from micrographs photographed at a magnification of ×40. Color information in the RGB images was discarded, and the images were converted to black and white files. Using NIH Image software, the intensity of fluorescence for each micrograph was measured by thresholding of the fluorescent structures (white). The scale ranged from 255 (white) to 0 (black). Measurements encompassed a total area of 1.8×10⁴ μm² for each excised tissue.

Additionally, apoptotic cell death was assessed with a terminal deoxynucleotidyltransferase-mediated dUTP nick-end labeling (TUNEL) reaction kit, according to the manufacturer's specifications (Roche Applied Science). The population of TUNEL-positive cells or TUNEL co-labeled oligodendrocytes in the optic nerve, retina, brain, and spinal cord were measured from micrographs photographed at a magnification of ×40. They encompassed a total area of 1.8×10⁴ μm² for each excised tissue. Labeled cells were counted manually.

Mitochondrial Membrane Potential: For visualization of mitochondrial membrane potential, optic nerve, retina, spinal cord, and brain specimens were immediately dissected out of 10 mice 3 days after antigenic sensitization for EAE, and 10 mice 6 days after EAE sensitization, and for comparisons to 10 control mice 3 days after inoculation with only Freund's adjuvant and 10 normal, unsensitized mice. The excised tissues were placed in tissue culture medium containing 0.3 μM MitoTracker Red (Molecular Probes, Eugene, Oreg.) in Dulbecco's modified Eagle's medium plus 10% fetal bovine serum for 20 min at 37° C., then washed in PBS, and processed for frozen sectioning and visualization of tissue fluorescence with a Leitz fluorescence microscope.

For quantitation of membrane potential, the optic nerve, retina, brain, and spinal cord were dissected out of 6 mice sensitized for EAE 3 days earlier for comparisons to 6 normal unsensitized mice. The excised tissues were incubated in 0.3 μM MitoTracker Red (Molecular Probes, Eugene, Oreg.) in Dulbecco's modified Eagle's medium plus 10% fetal bovine serum for 20 min at 37° C. and then washed in PBS. We quantified the mitochondrial membrane potential of the entire optic nerve from behind the eye to optic chiasm, the retina, the spinal cord, and brain using an Eclipse spectrofluorophotometer (Varian Instruments, Walnut Creek, Calif.). Fluorescence was normalized to protein content and measured using the DC protein assay kit (Bio-Rad).

Oxidative Phosphorylation Assay: The optic nerve, retina, brain, and spinal cord were dissected out of 6 mice sensitized for EAE 3 days earlier for comparisons to 6 normal unsensitized mice. Tissues were homogenized and resuspended in buffer (150 mM KCl, 25 mM EDTA, 0.1% bovine serum albumin, 10 mM potassium phosphate, 0.1 mM MgCl₂, pH 7.4). The rate of ATP synthesis of excised tissues was measured by chemiluminescence using a modified luciferin-luciferase assay in digitonin permeabilized tissues with the complex I substrates malate and pyruvate in real time using an Optocom I luminometer (MGM Instruments, Hamden, Conn.) and expressed per mg of protein. Cytoplasmic ATP synthesis was also measured after the addition of 10 ng/ml oligomycin to completely inhibit mitochondrial ATP production, thus giving the background level of ATP obtained by extra-mitochondrial substrate level phosphorylation.

Transmission and Light Microscopy: For light and transmission electron microscopy, optic nerve, retina, spinal cord, and brain specimens were dissected out of 10 mice 3 days after antigenic sensitization for EAE, and 10 mice 6 days after EAE sensitization, for comparisons to 10 normal unsensitized mice and 10 control mice inoculated with Freund's adjuvant and euthanized 3 days later. Immediately following euthanasia mice were perfused with fixative consisting of 4% paraformaldehyde and 2% glutaraldehyde in 0.1 M PBS buffer, pH 7.4. Tissues were further processed by immersion in 2.5% glutaraldehyde and then postfixed in 1% osmium tetroxide, 0.1 M sodium cacodylate-HCl buffer, pH 7.4. Tissues were then dehydrated through an ethanol series to propylene oxide, infiltrated, and embedded in epoxy resin that was polymerized at 60° C. overnight. Semithin longitudinal sections (0.5 μm to 1 μm) of the optic nerve head and retrobulbar nerve were stained with toluidine blue. In addition, ultrathin sections (90 nm) were placed on nickel grids for examination by transmission electron microscopy (model H7600; Hitachi, Tokyo, Japan) operating at 80 kV. For quantification of dissolution of mitochondrial cristae induced by early EAE, we analyzed 200 mitochondria from five animals sensitized for EAE and an equivalent number of mitochondria from five controls sensitized with the adjuvant. These 10 animals were euthanized 3 days after antigenic sensitization. Optic nerves were photographed at a magnification of ×10,000. Using NIH Image software the electron density of each mitochondrion was measured by manually tracing the silhouette of the outer membrane followed by thresholding of the electron dense cristae, including the mitochondrial membrane. Densities ranged from 255 to 0.

Statistical Analysis: Differences in the means between EAE and control groups were measured by Student's t test for unpaired data. Analysis of ATP synthesis and mitochondrial membrane potential was performed by analysis of variance. A difference in the means of 0.05 or less was considered statistically significant.

### Results

Nitration of CNS Tissues: We examined the optic nerves, retinas, brains, and spinal cords of 10 mice 3 days after sensitization for EAE with complete Freund's adjuvant and spinal cord emulsion for comparisons to specimens excised from 10 control mice 3 days after inoculation with only the Freund's adjuvant for ROS activity. At this early stage none of the animals exhibited any signs of paralysis from EAE (clinical stage 0).

As an initial gauge of ROS activity, we used the peroxynitrite-mediated nitration of tyrosine residues that was detected with an antibody directed against nitrotyrosine. Peroxynitrite is formed by the reaction of superoxide and nitric oxide. Using immunofluorescence cryomicroscopy, we detected nitrated proteins in the optic nerve, ganglion cell layer of the retina, brain, and spinal cord 3 days after sensitization for EAE. In contrast, the optic nerve, retina, brain, and spinal cord specimens of adjuvant-inoculated mice were unlabeled.

We excluded inflammatory cells, classically heralded as the mediators of tissue injury in EAE and MS, as the source of ROS activity by reacting EAE tissues with a pan-macrophage antibody followed by immunofluorescence microscopy and by light microscopic examination of toluidine blue-stained tissues. No inflammatory cells were detected in the optic nerve, brain, or spinal cord of mice sensitized for EAE 3 days earlier. As a positive control we examined tissue specimens available from our other experiments that were from animals sensitized for EAE 30 days earlier. They tested positive for inflammatory cells. Therefore, the origin of the nitrotyrosine immunofluorescence labeling in the 3-day EAE specimens appeared to be the CNS tissue itself.

Mitochondrial Protein Nitration: Because mitochondria are the primary source of cellular ROS, we probed them next. We isolated mitochondria from the optic nerves, retinas, brains, and spinal cords of 20 mice euthanized 3 days after sensitization for EAE and 20 mice euthanized 6 days after EAE sensitization for comparisons to controls consisting of 10 unsensitized animals and 10 mice inoculated with complete Freund's adjuvant and sacrificed 3 days later. Using immunoblotting, we then probed the mitochondrial isolates for peroxynitrite-mediated nitration of tyrosine residues with the nitrotyrosine antibody. We found several nitrated mitochondrial protein bands in the EAE central nervous system but not in the control specimens. Next, we attempted to determine which proteins were specifically inactivated in the mitochondria of EAE animals.

Identification of Nitrated Proteins: We identified the nitrated mitochondrial proteins using in situ trypsin digests of the excised nitrated protein bands followed by mass spectroscopy. When the resulting peptide fingerprints were compared with the protein sequence, the highest match was for mitochondrial heat shock protein 70 (mtHsp70). Protein data base sequence analysis of the other peptide fingerprints obtained included two respiratory chain complexes. They were identified as the NADPH-ubiquinone oxidoreductase B14 subunit of complex I (NDUFA6) and cytochrome c oxidase subunit IV. Consequently, oxidative damage to proteins in the mitochondrial respiratory chain was not uniform but affected subunits of complexes I and IV preferentially. Protein data base matches of another peptide fingerprint included the glycolytic enzyme glyceraldehyde 3-phosphate dehydrogenase (GAPDH).

Respiration Is Suppressed: To determine the impact of EAE on the primary function of mitochondria, we tested for generation of cellular ATP in six animals sensitized for EAE 3 days earlier and compared these to six unsensitized controls. The rates of mitochondrial ATP synthesis in the 3-day EAE optic nerve, retina, and brain were not significantly different from controls. However, in the spinal cords of mice sensitized for EAE 3 days earlier the rate of mitochondrial ATP synthesis was reduced by 79%, relative to controls (p<0.05).

Mitochondrial Membrane Potential Is Attenuated: Next, we examined the effect of EAE on mitochondrial membrane potential in 10 mice sensitized for EAE and 10 control mice inoculated with Freund's adjuvant. Each group was euthanized 3 days later, and the excised tissues were incubated with the membrane-sensing dye MitoTracker Red and prepared for cryomicrotomy. Fluorescence microscopy of the 3-day retrobulbar optic nerve sectioned just behind the eye revealed marked attenuation of labeling in the central region of the nerve. In contrast, the optic nerve of adjuvant-inoculated controls showed normal mitochondrial labeling of the entire optic nerve cross-section. Still, fluorospectrometric measurements of the entire optic nerve from the eye to the optic chiasm obtained from six animals sensitized for EAE 3 days earlier, relative to six controls, did not reflect differences in some optic nerve cross-sections visualized by microscopy.

The cell bodies of axons comprising the optic nerve reside in the ganglion cell layer of the retina. Microscopic examination of EAE-sensitized animals revealed some loss of MitoTracker Red labeling in the ganglion cell layer, relative to adjuvant inoculation. Fluorospectrometric measurements of the entire retina failed to show any difference between EAE and controls. In the EAE brain diminished MitoTracker Red labeling contrasted with the normal labeling of the adjuvant-inoculated animals. Quantitative measurements of the brain failed to detect any significant difference between EAE and adjuvant-inoculated control animals.

### Example 3

### Use of Mitochondrial Antioxidant Defenses for Rescue of Cells With a Leber Hereditary Optic Neuropathy-Causing Mutation

To explore a treatment paradigm for Leber hereditary optic neuropathy (LHON), we augmented mitochondrial antioxidant defenses to rescue cells with the G11778A mutation in mitochondrial DNA.

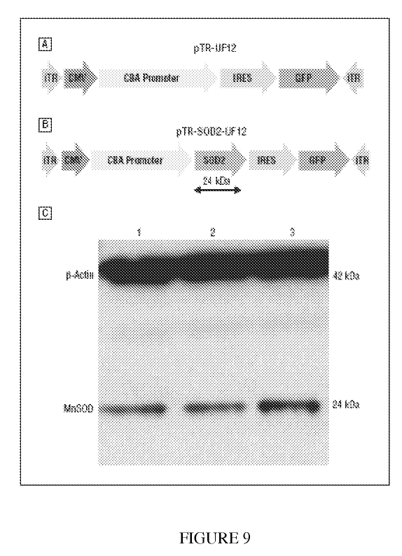

Superoxide Dismutase and Adeno-Associated Virus Vectors: We constructed an adeno-associated virus (AAV) vector using the AAV vector plasmid pTR-UF12 regulated by the 381-base pair (bp) cytomegalovirus enhancer immediate early gene enhancer and the 1352-bp chicken β-actin promoter-exon1-intron1 driving expression of the human mitochondrial superoxide dismutase (SOD2) complementary DNA (FIGS. 9A and 9B). This plasmid was linked to green fluorescent protein (GFP) via a 637-bp poliovirus internal ribosomal entry site. The SOD2-containing plasmid and the parent pTR-UF12 plasmid were amplified and purified by means of cesium chloride gradient centrifugation and then packaged into AAV-2 capsids by transfection into human 293 cells using standard procedures. Genome titers of the recombinant AAV were determined using real-time polymerase chain reaction and assayed for infectious particles. Each virus preparation contained 10¹¹ to 10¹² vector genome particles/mL and 10⁹ to 10¹⁰ infectious center U/mL.

Cell Culture and Infection: Homoplasmic 143B osteosarcoma cells (cybrids) containing 100% mutated (11778A) mtDNA were grown in Dulbecco's modified eagle medium (Fisher Scientific, Hampton, N.H.) supplemented with 10% heat-inactivated fetal bovine serum and 1% penicillin streptomycin (Sigma-Aldrich Corp, St Louis, Mo.) at 37° C. with 5% carbon dioxide. The cybrids were created by fusion of enucleated cells from patients with mutated mtDNA, in this case the G11778A mutation, with osteosarcoma (143B.TK)-derived human cells containing wild-type mtDNA cells that were depleted of their mtDNA by chronic exposure to ethidium bromide (ρ0 cells). The LHON cybrids were seeded in two 6-well or two 96-well dishes. For AAV infections, cybrid cells at approximately 50% confluency were infected at multiplicities of infection of 5000 viral particles per cell, one 6-well dish or one 96-well dish with AAV-SOD2, and one 6-well dish or one 96-well dish with AAV-GFP. Two days after the AAV infections, the high-glucose medium was replaced with glucose-free galactose medium (Guy J, et al., Ann Neurol. 2002; 52:534-542). This selective medium forces the cells to use oxidative phosphorylation to produce adenosine triphosphate. After 2 days of growth in glucose-deficient galactose medium, the SOD2-infected cells from each of 6 wells and the GFP-infected cells from each of 6 wells were trypsinized and counted using an automated particle counter (Z-100; Coulter Diagnostics, Hialeah, Fla.). After 3 days of growth in glucose-deficient galactose medium, the SOD2-infected cells from each of 10 wells and the GFP-infected cells from each of 10 wells were trypsinized and counted.

Detection of SOD2 Expression: Two days after AAV infections, we harvested AAV-SOD2-transfected cybrids, control cells infected with AAV-GFP (Fernandez-Vizarra E, et al. Methods. 2002; 26:292-297), or LHON cells that were not exposed to either AAV. Briefly, this involved washing the trypsinized cells in cold phosphate-buffered saline solution. Cells were then manually homogenized and stored at −80° C. for later analysis. For immunodetection, 15 μg of total protein was separated on a 10% sodium dodecyl sulfate-polyacrylamide gel and electrotransferred to a polyvinylidene fluoride membrane (BioRad Laboratories, Hercules, Calif.). The protein content of the samples was measured using a DC protein assay (BioRad Laboratories). We immunostained the membrane with polyclonal anti-SOD2 antibodies (Stressgen Bioreagents, Victoria, British Columbia) and then goat anti-rabbit IgG horseradish peroxidase-conjugated secondary antibodies (Sigma-Aldrich Corp). We detected complexes using the enhanced chemiluminescence system (Amersham Pharmacia Biotech, Piscataway, N.J.). Antimouse β-actin antibody was used as an internal control for protein loading.

Detection of Superoxide: We used the fluorescent probe dihydroethidium (DHE) to detect intracellular superoxide (Molecular Probes, Eugene, Ore). Superoxide oxidizes the weakly blue fluorescent DHE to a bright red fluorescent signal. Cybrids were seeded into 48 wells of the 96-well plates. Cells in 24 wells were transfected with SOD2, and cells in the other 24 wells were transfected with GFP. Two days later, the medium was replaced with glucose-free galactose medium. After 24 or 48 hours, cells were incubated with 1 μM DHE for 20 minutes at 37° C. They were washed and then observed under a fluorescence microscope (Leitz, Wetzlar, Germany).

The intensity of fluorescence was quantitated using a fluorophotometer (Eclipse; Varian Medical Systems, Palo Alto, Calif.) with excitation at 480 nm and emission at 560 nm (red). Wells were counted in duplicate or greater. Protein content of the samples was measured using the DC protein assay (BioRad Laboratories), and the intensity of fluorescence was adjusted to the sample protein content. We selected DHE not only because of its specificity for detection of intracellular superoxide but also because other commercially available fluorophores such as dichlorodihydrofluorescein have a green emission similar to that of GFP and may interfere with detection of the oxidized green fluorescence of dichlorodihydrofluorescein. In contrast, the peak of red fluorescent DHE oxidized by superoxide and used herein was easily distinguished from the other emission at 520 nm from the green fluorescence of GFP.

Detection of Apoptosis: Cybrids were seeded into 48 wells of the 96-well plates. Cells in 24 wells were transfected with AAV-SOD2, and cells in the remaining 24 wells were transfected with AAV-GFP. Two days later, the high-glucose medium was exchanged for glucose-free galactose medium. After 1 day (24 wells) and 2 days (24 wells) in this restrictive medium, apoptotic cell death was assessed with a TUNEL (terminal deoxynucleotidyl transferase-mediated biotin-deoxyuridine triphosphate nick-end labeling) reaction kit, according to the manufacturer's specifications (Roche Diagnostics Corp, Indianapolis, Ind.). The red TUNEL-positive cells (emission, 560 nm) were visualized and quantitated as described for superoxide.

Statistical Analysis: We compared the AAV-SOD2-transfected cells with controls inoculated with AAV-GFP. Statistical analysis was performed by analysis of variance. P<0.05 was considered significant.

### Results

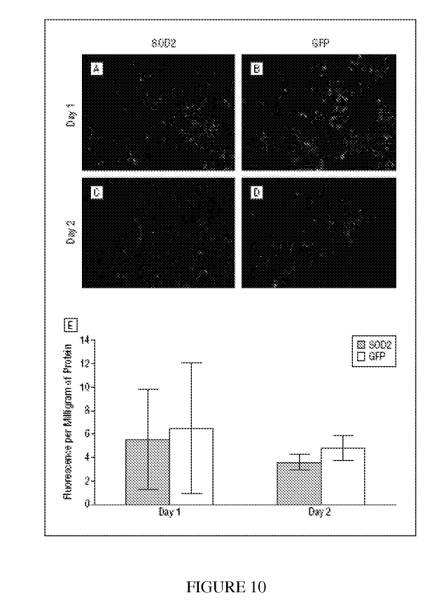

Increase of SOD2 and Decrease of Superoxide with AAV-SOD2: Immunoblots of AAV-SOD2-infected LHON cells showed increased manganese SOD expression relative to the control uninfected cybrids and those infected with AAV-GFP (FIG. 9C). Fluorescence micrographs confirmed a decrease in superoxide-induced fluorescence following AAV-SOD2 infection. Treatment with AAV-SOD2 decreased superoxide-induced DHE fluorescence in LHON cells after 1 day (FIG. 10A) or 2 days (FIG. 10C) in the restrictive medium, relative to infection with AAV-GFP (FIGS. 10B and 10D). After 1 day of growth in the glucose free galactose medium, quantitative analysis of the emission at 560 nm that was distinct from the green emission of GFP at 520 nm revealed that superoxide induced DHE fluorescence decreased 15% relative to AAV infection with AAV-GFP (FIG. 10E). This difference was not statistically significant. However, after 2 days of growth in this restrictive medium, superoxide-induced DHE fluorescence decreased 26% relative to the LHON cells infected with the control AAV. This difference was significant (P=0.003). Clearly, SOD2 suppressed cellular production of superoxide.

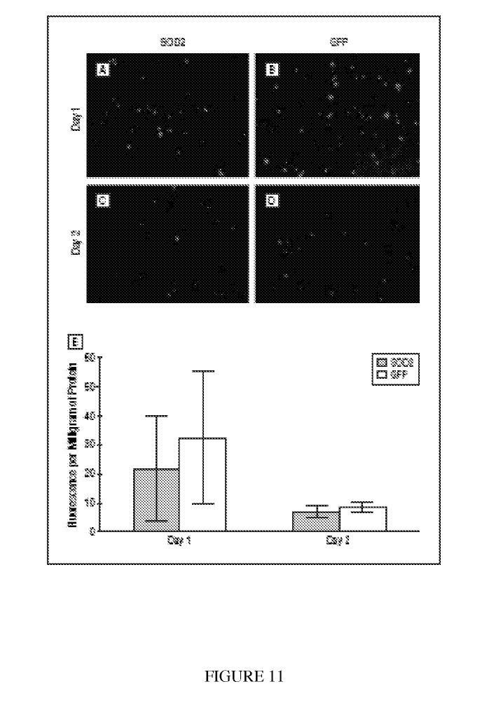

Suppression of Apoptosis with AAV-SOD2: Because mitochondrial oxidative stress is closely linked to apoptotic cell death, we assayed for TUNEL-positive cells as early as 1 day after growth in the galactose medium. Treatment with AAV-SOD2 decreased TUNEL-positive LHON cells after 1 day (FIG. 11A) or 2 days (FIG. 11C) in the restrictive medium, relative to infection with AAV-GFP (FIGS. 11B and 11D). Quantitative analysis revealed that, relative to the control AAV infection, the intensity of TUNEL fluorescence was diminished by 34% (not significant) after 1 day and 21% (P=0.048) with SOD2 infection after 2 days in the galactose medium (FIG. 11E). Clearly, SOD2 infection protected LHON cells against apoptotic cell death.

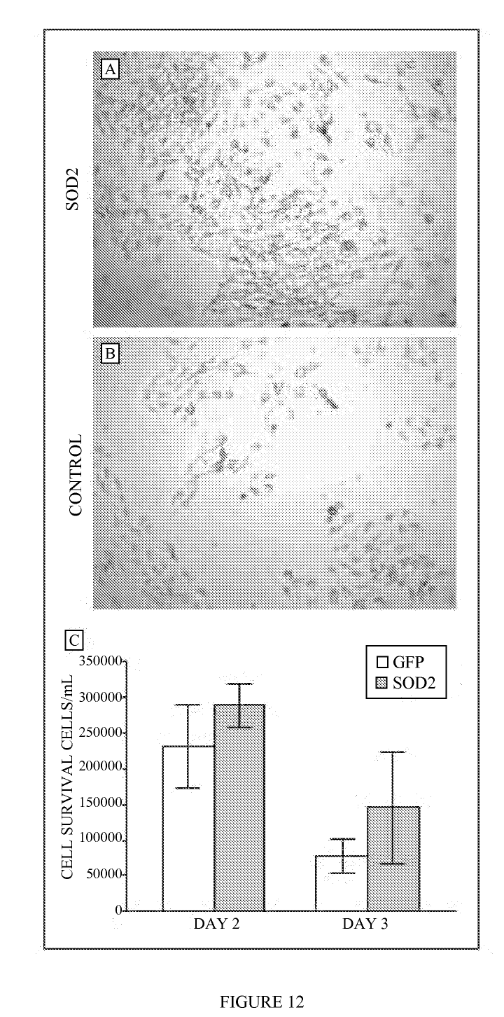

AAV-SOD2 Increases LHON Cell Survival: Reducing apoptotic cell death by protection against mitochondrial oxidative stress with AAV-SOD2 increased the survival of LHON cybrids. After 2 days of growth in the galactose medium, we found that LHON cell survival increased by 25% with AAV-SOD2 infection relative to the control infection with AAV-expressing GFP(P=0.05) (FIGS. 12A-C). Although the population of cells dwindled relative to 2 days of growth in the galactose medium, after 3 days of growth in this restrictive medium, we found that AAV-SOD2 increased LHON cell survival by 89% relative to the controls (P=0.006) (FIG. 12C). Clearly, increasing mitochondrial antioxidant defenses rescued LHON cells.

Summary: Our findings show that the superoxide anion is involved in LHON cell death and suggest that increasing mitochondrial antioxidant defenses maybe a potential treatment for LHON. Reactive oxygen species that include superoxide anion, hydrogen peroxide, nitric oxide, and peroxynitrite are major initiators of the apoptotic pathway leading to cell death in LHON cells. Although tissue levels of SOD2 expression and activity in the optic nerves of patients with LHON have yet to be determined, a decrease in mitochondrial SOD activity has been detected in the LHON cybrid cell line. Mitochondria mitigate oxygen toxicity predominantly via enzymatic antioxidants that include SOD and glutathione peroxidase. Lowered levels of mitochondrial SOD activity likely increase cellular injury and induce optic neuropathy in mitochondrial disorders, particularly those like LHON that are related to a loss of complex I activity.

Bolstering anti-reactive oxygen species defenses may suppress the death of retinal ganglion cells in LHON. Rescue of our animal model of complex I deficiency with SOD2 suggests that antioxidant gene therapy may be useful for patients with complex I deficiencies such as LHON. In that model system, suppression of reactive oxygen species inhibited apoptotic death of retinal ganglion cells, a phenomenon that is also involved in the pathogenesis of disease caused by the mutated human ND4 complex I subunit gene. Apoptotic cell death associated with complex I impairment induced by rotenone can also be blocked by overexpression of SOD2, further supporting our work.

Treatment options for patients with LHON and those with other mitochondrial disorders are limited at present. The most direct approach to treatment would be to correct the mutated mitochondrial DNA. Although genes have been inserted into the nucleus and cytoplasm through the use of vectors, the technology to introduce a gene into the mitochondria is not yet possible. Because it is expression of the mutant complex I subunit at the protein level that causes the biochemical defect of LHON, an alternative and feasible approach is to import a normal protein allotopically into the mitochondria to complement the defective protein encoded by the mutated mtDNA. We have shown allotopic rescue of this same LHON cell line with mutated G11778A mtDNA supports this form of intervention. However, a different allotopic construct would be needed for the 3 mitochondrial genes containing mutations in ND1, ND4, or ND6 responsible for 85% of LHON cases.

Studies showing subtle retinal and optic nerve injury in families harboring the G11778A mtDNA mutation suggest that treatment may be necessary before symptoms actually develop. Nevertheless, many patients with LHON are found at the initial examination to have optic disc edema and predominantly unilateral visual loss. Thus, there is a window of opportunity of several months for prophylactic intervention in the fellow eye with SOD2 gene therapy before it too loses vision. Still, the early retinal changes detected in LHON carriers before apoplectic visual loss suggest that this approach may have the best chance for success if it is initiated at the earliest stages of disease. The aim would be to reduce the accumulation of optic nerve damage so that injury does not progress to a point beyond which loss of function becomes irreversible.

1. An adeno-associated AAV vector comprising: a cytomegalovirus enhancer, a promoter, a human mitochondrial superoxide dismutase (SOD2) gene, an internal ribosome entry site (IRES) and a detectable marker gene.

2. The adeno-associated AAV vector of claim 1, wherein the cytomegalovirus enhancer, the promoter, the human mitochondrial superoxide dismutase (SOD2) gene, the internal ribosome entry site (IRES) and the detectable marker gene are interposed between inverted terminal repeat sequences.

3. The adeno-associated AAV vector of claim 1, wherein the vector is an AAV vector comprising Rep, Cap, inverted terminal repeat (ITR) sequences and the AAV is selected from the group consisting of AAV-1 to AAV-9 serotypes.

4. The adeno-associated virus vector of claim 1, wherein the promoter is a hybrid cytomegalovirus/β-actin promoter.

5. A cell comprising an adeno-associated AAV vector comprising: a cytomegalovirus enhancer, a promoter a human mitochondrial superoxide dismutase (SOD2) gene, an internal ribosome entry site (IRES) and a detectable marker gene; wherein the vector is an AAV vector comprising Rep, Cap, inverted terminal repeat (ITR) sequences and the AAV is selected from the group consisting of AAV-1 to AAV-9 serotypes.

6. The cell of claim 5, wherein the cytomegalovirus enhancer, the promoter, the human mitochondrial superoxide dismutase (SOD2) gene, the internal ribosome entry site (IRES) and the detectable marker gene are interposed between the inverted terminal repeat sequences.

7. The cell of claim 5, wherein the cell is a mammalian cell.

8. The cell of claim 5, wherein the cell is isolated from a patient suffering from a disease or disorder associate with abnormal levels of reactive oxygen species comprising: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

9. A method of inhibiting mitochondrial oxidative stress in a cell or animal, comprising:
administering to a cell or animal a nucleic acid comprising an adeno-associated AAV vector comprising: a cytomegalovirus enhancer, a promoter, a human mitochondrial superoxide dismutase (SOD2) gene, an internal ribosome entry site (IRES) and a detectable marker gene; and;
expressing the human mitochondrial superoxide dismutase (SOD2) gene in the cell or animal; and,
inhibiting mitochondrial oxidative stress in a cell or animal.

10. The method of claim 9, wherein the nucleic acid is administered in an amount sufficient to inhibit mitochondrial oxidative stress in a cell or animal between about 50% up to 100% as compared to an abnormal cell; and, normalizing reactive oxygen species in the abnormal cell to normal cell levels.

11. The method of claim 9, wherein the abnormal cell comprises a cell isolated from a patient that is suffering from a disease or condition caused by reactive oxygen species comprising inflammation, shock, cancer and ischemia/reperfusion injury.

12. The method of claim 9, wherein the abnormal cell is isolated from a patient suffering from a disease or disorder associate with abnormal levels of reactive oxygen species comprising: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

13. A method of treating a patient suffering from a disease or disorder associated with enhanced mitochondrial oxidative stress comprising:
administering to a patient a nucleic acid comprising an adeno-associated AAV vector comprising: a cytomegalovirus enhancer, a promoter, a human mitochondrial superoxide dismutase (SOD2) gene, an internal ribosome entry site (IRES) and a detectable marker gene; and;
expressing a human mitochondrial superoxide dismutase (SOD2) gene in a cell or patient; and,
treating the patient suffering from a disease or disorder associated with enhanced mitochondrial oxidative stress.

14. The method of claim 13, wherein the nucleic acid is administered in an amount sufficient to inhibit mitochondrial oxidative stress in the patient between about 50% up to 100% as compared to an abnormal cell; and, normalizing reactive oxygen species in the abnormal cell to normal cell levels.

15. The method of claim 13, wherein the disease or disorder associate with abnormal levels of reactive oxygen species comprises: Leber's hereditary optic neuropathy (LHON), optic neuritis, multiple sclerosis, ischemic reperfusion injury, inflammatory diseases, systemic lupus erythematosis, myocardial infarction, stroke, traumatic hemorrhage, spinal cord trauma, Crohn's disease, autoimmune diseases, cataract formation, uveitis, emphysema, gastric ulcers, oxygen toxicity, neoplasia, undesired cell apoptosis, and radiation sickness.

16. A method of introducing nucleic acid molecules into mitochondria (intra-mitochondrially) comprising:
administering to a cell or patient a composition comprising a vector encoding a cytomegalovirus enhancer, a promoter, a human mitochondrial superoxide dismutase (SOD2) gene, an internal ribosome entry site (IRES) and a detectable marker gene;
introducing nucleic acid molecules into mitochondria.

17. The method of claim 16, wherein the cytomegalovirus enhancer, the promoter, the human mitochondrial superoxide dismutase (SOD2) gene, the internal ribosome entry site (IRES) and the detectable marker gene are interposed between inverted terminal repeat sequences.

18. The method of claim 17, wherein the vector is an AAV vector comprising Rep, Cap, inverted terminal repeat (ITR) sequences and the AAV is selected from the group consisting of AAV-1 to AAV-9 serotypes.

19. The method of claim 16, wherein the promoter is a hybrid cytomegalovirus/β-actin promoter.

20. An Adenovirus Associated Virion comprising a cytomegalovirus enhancer, a promoter, a human mitochondrial superoxide dismutase (SOD2) gene, an internal ribosome entry site (IRES) and a detectable marker gene.

21. The Adenovirus Associated Virion of claim 20, wherein the cytomegalovirus enhancer, the promoter, the human mitochondrial superoxide dismutase (SOD2) gene, the internal ribosome entry site (IRES) and the detectable marker gene are interposed between inverted terminal repeat sequences.

22. The Adenovirus Associated Virion of claim 20, wherein the vector is an AAV vector comprising Rep, Cap, inverted terminal repeat (ITR) sequences and the AAV is selected from the group consisting of AAV-1 to AAV-9 serotypes.

23. The Adenovirus Associated Virion of claim 20, wherein the promoter is a hybrid cytomegalovirus/β-actin promoter.
